(19) United States
(12) Patent Application Publication
(10) Pub. No.: US 2020/0085306 A1
(43) Pub. Date: Mar. 19, 2020

(54) ELECTRONIC DEVICE FOR MEASURING PHYSIOLOGICAL INFORMATION AND A METHOD THEREOF

(71) Applicant: Amorv (IP) Company Limited, Kowloon (HK)

(72) Inventors: Kwan Wai TO, Kowloon (HK); Wang Kin FUNG, Kowloon (HK); Luis NG, Kowloon (HK); Ngok Man SZE, Kowloon (HK); Chi Tin LUK, Kowloon (HK); Wenbo GU, Kowloon (HK)

(21) Appl. No.: 16/692,078

(22) Filed: Nov. 22, 2019

Related U.S. Application Data
(63) Continuation-in-part of application No. 16/317,573, filed on Jan. 14, 2019.
(60) Provisional application No. 62/770,851, filed on Nov. 23, 2018.

(51) Int. Cl. A61B 5/00; A61B 5/022

(52) U.S. Cl. CPC A61B 5/0064; A61B 5/681; A61B 5/022; A61B 5/684; A61B 5/6843

(57) ABSTRACT

An electronic device measures physiological information of a living subject. The electronic device includes a sensor assembly, a first driving unit with an electromagnetic structure and a second driving unit. The first driving unit drives the sensor assembly to scan the living subject's skin along a scan path in a non-contact way to determine a measuring position. The second driving unit drives the sensor assembly to move towards and contact the living subject's skin to measure the physiological information based on the measuring position. In a typical embodiment, the sensor is configured under a measurement surface where a wrist of a user is put on. The sensor scans the user's wrist along a scan path under the wrist in a non-contact way and move upwards to contact the user's wrist for measuring the physiological information based on the scanning result.

### TECHNICAL FIELD

This invention relates to an electronic device that measures physiological information of a living subject.

### BACKGROUND ART

Nowadays, technology integrated with health tools are becoming a popular trend within the healthcare industry and are being used on a more regular basis. Many of the electronic devices are providing a plethora of health data from the growing roster of available tools that can be used by consumers for both personal and clinical decisions. Generally, the electronic devices with health tools could measure heart rate (HR), heart rate variability (HRV), blood pressure, temperature, motion, and/or other biological information of the user via a noninvasive method.

In one application field, an electronic device is designed to measure health data of a user, e.g., heart rate and blood pressure, via the blood vessel of the wrist. A cuff-type wrist blood pressure meter occludes all blood vessels around the wrist to measure the blood pressure. Hence, it cannot be used to measure continual blood pressure. In order to measure continual blood pressure, some electronic devices measure photoplethysmography (PPG) signals and electrocardiogram (ECG) signals to calculate pulse transit time (PTT) and estimate blood pressure accordingly. However, frequent calibration is needed for PTT-based blood pressure estimation. Also, it is inconvenient to measure both PPG and ECG.

In view of demand for measuring health data, improvements that provide an accurate and compact electronic device for continual blood pressure measurement are desired.

### SUMMARY OF THE INVENTION

The present invention is directed to an electronic device that measures physiological information of a living subject. In one example embodiment, the electronic device includes a sensor assembly, a first driving unit with an electromagnetic structure and a second driving unit. The first driving unit drives the sensor assembly to scan the living subject's skin along a scan path in a non-contacting way to determine a measuring position. The second driving unit drives the sensor assembly to move towards and contact the living subject's skin to measure the physiological information based on the measuring position. Other example embodiments are discussed herein.

### BRIEF DESCRIPTION OF THE DRAWINGS

FIG. 1 illustrates a preferred location on a wrist of a user 100 for measuring the blood pressure according to one example embodiment.

FIG. 2 illustrates a block diagram of an electronic device 200 for healthcare, according to one example embodiment.

FIG. 3 illustrates a schematic drawing of an electronic device 300 for healthcare, according to one example embodiment.

FIG. 4 illustrates a schematic drawing of an electronic device 400 for detecting the blood pressure at an internal side of a user's wrist, according to one example embodiment.

FIG. 5 shows a top view of the sensor assembly 206 used in the electronic device 200, according to one example embodiment.

FIGS. 6A and 6B show an operating mechanism of the electronic device with the sensor assembly 206 on the user's wrist, according to one example embodiment.

FIG. 7A shows waveforms of a reflected optical signal and pressure pulse signal received by the sensor assembly 206 when it is in touch with the user's wrist as illustrated in FIG. 6A, according to one example embodiment.

FIG. 7B shows waveforms of a reflected optical signal and pressure pulse signal received by the sensor assembly 206 when it is pressed against the wrist surface as illustrated in FIG. 6B, according to one example embodiment.

FIG. 8A illustrates an exemplary schematic structure of the electronic device 200 with a membrane unit, according to one example embodiment.

FIG. 8B shows the new membrane section 873 of the membrane unit from a bottom view, according to one example embodiment.

FIG. 8C shows the electronic device 200 with the membrane unit contacting the user's skin, according to one example embodiment.

FIG. 9A shows a top view of a sensor assembly 906 with a coating layer, according to one example embodiment.

FIG. 9B shows a cross section view (from AA′ direction of FIG. 9A) of the sensor assembly 906 with the coating layer, according to one example embodiment.

FIG. 10A shows a movable frame being worn on a user's wrist via a wristband, according to one example embodiment.

FIG. 10B shows a portable device that disposes on the user's wrist and coupled to the movable frame for measuring the health information of the user, according to one example embodiment.

FIG. 11 illustrates a measurement relationship between the sensed signal, the scanning trace and the skin contour, according to one example embodiment.

FIG. 12 illustrates a flowchart of predicting the measuring position via a prediction algorithm, according to one example embodiment.

FIG. 13 illustrates a method of applying an electronic device to a user, in according to one example embodiment.

FIGS. 14A and 14B show a schematic drawing of a portable device in operating mode for measuring physiological information of a user.

FIG. 15 shows a measuring band wearing on the wrist for the measurement operated by the portable device.

FIG. 16 shows a cross-sectional view of the wristband being magnetically coupled with the device during the operation.

FIGS. 17a and 17b respectively illustrate schematic drawings of a top view and a perspective view of the device shown in FIGS. 14A and 14B.

FIG. 18 depicts an alternative embodiment in which a one-piece ferromagnetic component may be separated into several blocks applied on the wristband.

FIGS. 19A and 19B illustrate the instruction indicators on the wristband.

FIG. 20 shows a measurement module 2000 of the device, in accordance with another embodiment of the present invention.

FIG. 21 illustrates a locking mechanism of the device for locking the wristband during the operation, in accordance with another embodiment of the present invention.

FIG. 22 illustrates a schematic drawing of the operating mode of a sensor in the device for detecting the vital signs on the user's wrist.

FIG. 23 is a schematic depiction of a mechanical structure of a sensor.

FIGS. 24A-24B schematically illustrate a mechanical structure of a sensor.

FIG. 25 shows a schematic drawing of a portable device with peripheral components for measuring physiological information of a user.

FIG. 26 shows an operation flowchart of a portable device for measuring physiological information of a user.

FIG. 27 is an example showing a detailed mechanical structure among a leverage unit, a resisting element and the supporting element within the mechanical structure of the sensor in FIGS. 24A-24B.

FIG. 28 shows an operation flowchart of a portable device for measuring physiological information of a user.

### DETAILED DESCRIPTION

Reference will now be made in detail to the embodiments of the present invention. While the invention will be described in conjunction with these embodiments, it will be understood that they are not intended to limit the invention to these embodiments. On the contrary, the invention is intended to cover alternatives, modifications and equivalents, which may be included within the spirit and scope of the invention as defined by the appended claims.

Furthermore, in the following detailed description of the present invention, numerous specific details are set forth in order to provide a thorough understanding of the present invention. However, it will be recognized by one of ordinary skill in the art that the present invention may be practiced without these specific details. In other instances, well known methods, procedures, components, and circuits have not been described in detail as not to unnecessarily obscure aspects of the present invention. In the light of the foregoing background, it is an object of the present invention to provide an electronic device for monitoring the health status of the user.

In one example embodiment, an electronic device for healthcare is, but not limited to, a wrist-worn device that measures the health data, e.g., heart rate, heart rate variability, blood pressure, blood oxygen saturation, and/or stress, of a user. In one embodiment, the electronic device is a wristband that is rigid or flexible to be worn on the wrist and can have various shapes and sizes without departing from the scope of example embodiments. Other example embodiments can be worn on an arm, neck, leg, ankle, or other part of the human body.

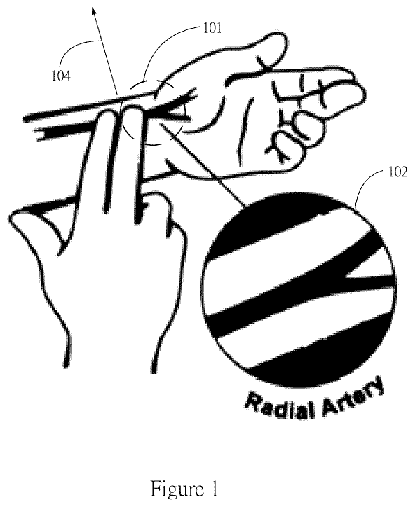

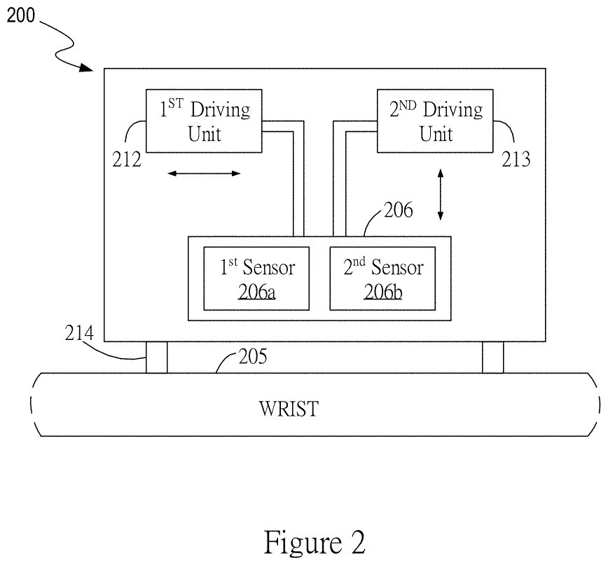

FIG. 1 illustrates a location on a wrist 100 for measuring the blood pressure, according to one example embodiment. The electronic device includes a pressure sensor (shown in FIG. 2), which is to be located near an artery of the wrist, for measuring the blood pressure. More specifically, the pressure sensor is located near a radial artery superficially above the distal end of the radial bone, as exemplarily specified by a dot line circle 101. An enlarged image 102 of the radial artery within the circle 101 shows more details thereof, in which the pressure sensor is located within a neighboring region of the radial artery. When the radial artery is compressed against the radial bone by the pressure sensor, the pulse can be clearly sensed at the wrist where it is covered by thin skin and tissue. As such, a target neighboring region of the radial artery of the wrist, which is exemplarily specified as the circle area 101 to secure an accurate measurement, needs to be determined.

In some cases, a pressure sensor array is used to detect multiple pulse signals within a certain area, e.g., the circle area 101, and to select a largest one of which the location is near the radial artery of the wrist for accurately measuring the health data, e.g., heart rate and blood pressure, of the user. However, the pressure sensor array is bulky and expensive. In some cases, a motor is used to move a pressure sensor along the wrist surface in a predetermined direction, e.g., a direction 104 perpendicular to the artery direction as shown in FIG. 1, to detect multiple pulse signals within the predetermined direction. Similarly, a largest detected signal is selected of which the corresponding location is determined for measuring the health data of the user. However, during the movement, since the pressure sensor is in touch with the skin surface, the motor with enough torque is needed to overcome the friction between the sensor and the skin surface. Step motors or DC motors are used for driving the pressure sensor to sweep along the wrist surface to obtain multiple pulse signals for identifying the optimal measuring position where an artery is predicted to lie thereunder. However, the step or DC motors are bulky which make the whole electronic device not compact and inconvenient for long-term use.

In order to overcome the above problems, a non-contact sensor, e.g., a wireless wave sensor, is adopted to move along the wrist surface to sense the physiological information of the user at multiple positions, that is, to scan the wrist skin, without, at least partially, contacting the skin surface (in a non-contact way) and determine a measuring position based on the sensed physiological information, according to one example embodiment. In one example embodiment, the wireless wave sensor includes electromagnetic or mechanical wave sensor that is able to emit and detect electromagnetic or mechanical wave. In one example embodiment, the wireless wave sensor is an optical sensor. In one example embodiment, a non-contact scan region is around 15-20 mm. In one example embodiment, the measuring position is near to, at least within an acceptable neighboring range of, the target blood vessel under the wrist surface. In one example embodiment, the non-contact sensor emits a signal, e.g., an optical or ultrasound signal, toward the wrist surface and detects the signal reflected from the wrist during the movement. Based on the detected signal, the target measuring position is identified. A pressure sensor is then used to sense the physiological information based on the identified measuring position. In one embodiment, the accuracy of the measuring position identified by non-contact scanning is around 3 mm and then the pressure sensor needs to fine-tune the measuring position by sensing pressure pulse signals at multiple positions surrounding the identified measuring position and determine a more accurate position based on the sensed pressure pulse signals. In another embodiment, the accuracy of the measuring position identified by non-contact scanning has been increased to within 1 mm according to a prediction algorithm. In this case, the pressure sensor can directly sense the blood pressure at the measuring position identified by the non-contact scanning process. In one embodiment, since during the scanning process, the non-contact sensor is above the skin surface without, at least partially, contacting the skin surface, the torque needed to drive the non-contact sensor to move along the wrist surface is significantly reduced, as compared with the torque needed to drive the pressure sensor to contact and move along the skin surface. Under such condition, a more compact driving unit, e.g., Voice Coil Motor (VCM), can be used to move the non-contact sensor along the wrist surface. The dimension of the VCM is much smaller than that of the other mechanical/electrical motor, e.g., step or DC motors. Furthermore, the VCM can control the tilting angle of the sensors mounted on the motor so that more accurate measurement can be achieved.

FIG. 2 illustrates a block diagram of an electronic device 200 for healthcare, according to one example embodiment. As shown in FIG. 2, when the electronic device 200 is worn on a user's wrist 216, it is mounted on the inner side 205 of the wrist 216 via the supporting unit 214. The electronic device 200 includes a sensor assembly 206 that is used for sensing the physiological information, e.g., pulse rate, blood pressure and blood oxygen saturation information, of the living object; a first driving unit 212, that drives the sensor assembly 206 to scan the wrist surface 205 within a predetermined region to detect the measuring position for measuring the health data of the user; and a second driving unit 213 that drives the sensor assembly 206 to move in a direction perpendicular or substantially perpendicular to the wrist surface 205 in order to keep a certain distance between the skin and the sensor assembly 206 when doing non-contact scanning and drives the sensor assembly 206 to contact and press against the wrist skin when sensing blood pressure pulsation and a photoplethysmography waveform.

In one example embodiment, the sensor assembly 206 includes a first sensor 206a and a second sensor 206b, wherein the first sensor 206a is an optical sensor that detects the artery position of the wrist in a non-contact way, and the second sensor 206b is a pressure sensor which is driven by a hold-down force to contact and press against the wrist surface 205 for fine-tuning measurement location and measuring the pressure against the wall of the artery. In one embodiment, the first sensor 206a emits light toward the wrist 216 and detects the light reflected from the wrist 216 while moving along a predetermined path, so as to determine the artery position based on the detected result. In one embodiment, the first sensor 206a and the second sensor 206b are integrated in the sensor assembly 206 and moved together in a first and second directions by the first driving unit 212 and the second driving unit 213. In an alternative example embodiment, the first sensor 206a and second sensor 206b are separated units. Under such condition, the first sensor 206a will be driven to non-contactingly (i.e., without physically contacting) scan the wrist surface for determining the measuring position and the second sensor 206b will be driven to press against the skin to sense the blood pressure at the measuring position. However, for easy illustration and understanding, the embodiment that the first and second sensors 206a and 206b move together with the sensor assembly 206 will be used for later description and it is understood by people having ordinary skill in the art that other embodiments, e.g., the first and second sensors 206a and 206b are separated and moved independently from each other, could be also applied to the following description with reasonable changes.

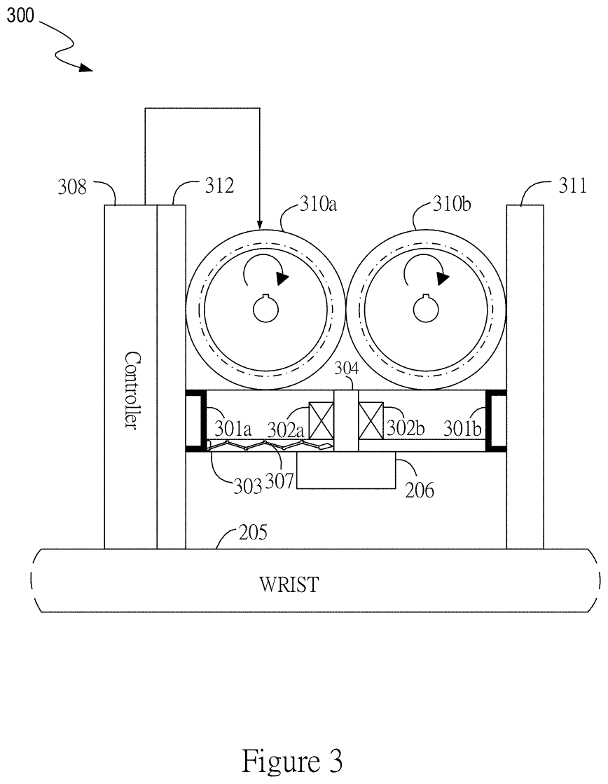

FIG. 3 illustrates a schematic drawing of an electronic device 300 for healthcare, in accordance with one example embodiment. FIG. 3 is described in combination with FIG. 2. Elements with the same or similar reference numerals have the same or similar structure/function thereof in previous figures. In the electronic device 300 of FIG. 3, a guiding unit 314 couples to the sensor assembly 206 for guiding the sensor assembly 206 to scan the wrist surface 205 in a non-contact (i.e., without physically contacting) way. In one example embodiment, the guiding unit 314 comprises at least one guiding rail 303 and at least one moving element 304 moving along the guiding rail 303. In one example embodiment, the moving element 304 is a rolling or sliding element. As understood by one skilled in the art, the guiding unit 314 is not limited to the structure as illustrated in FIG. 3 and could have alternative configurations. For example, the guiding rail 303 could guide the moving element 304 to move in a straight or curved orientation. The guiding rail 303 and the moving element 304 could be of any shape/structure as long as it satisfies the guiding function. Furthermore, the first driving unit 212 from FIG. 2 (not specified in FIG. 3) is an electromagnetic motor that includes at least one magnet 301a, and at least one coil 302a that interacts with the magnet 301a for generating an electromagnetic force. The electromagnetic motor couples to the guiding unit 314 for driving the guiding unit 314 to guide the sensor along a predetermined scanning path above the skin surface. In one example embodiment, the first driving unit 212 is a VCM motor.

In one example embodiment, the magnet 301a is fixed and the coil 302a is movable and mounts to the moving element 304. When a current flows through the coil 302a, an electromagnetic force is generated between the magnet 301a and the coil 302a to enable the coil 302a, together with the moving element 304, to move toward or away from the magnet 301a along the guiding rail 303. In an alternative example embodiment, the coil 302a is fixed and the magnet 301a is movable and attached to the moving element 304 in a similar structure. Furthermore, an elastic unit 307, with one end being coupled to the moving element 304, provides a restoring force to the moving element 304. In one example embodiment, the elastic unit 307 is a spring. In one example embodiment, the elastic unit 307 has one end that is fixed to the magnet 301a and the other end is coupled to the moving element 304. Under a combined effect of the electromagnetic force and the restoring force, the guiding unit 314 could guide the sensor assembly 206 to move toward and stay steadily at a target position when the current flows through the coils 302a. When no current flows through the coils 302a, the restoring force of the elastic unit 307 will bring the sensor assembly 206 back to its initial position.

In one example embodiment, a friction force between the guiding rail 303 and the moving element 304 is predefined to reduce the shift and improve the stability of the sensor assembly 206 while staying at the targeted position. In another example embodiment, two or more sets of the magnet and coil 301a/302a and 301b/302b are disposed at two sides of the moving element 304 in order to provide pushing/pulling force at the two sides of the moving element 304 for enhancing movement control and improving stability. One of ordinary skill in the art may appreciate that details of the electronic device as discussed therein are merely examples. Other embodiments and details can be provided by the electronic device without departing from the scope of this invention. For example, the elastic unit 307 could be configured in any format at any place as long as it satisfies the requirement of providing a restoring force that corresponds to an electromagnetic force to bring the sensor assembly 206 back to its initial position when the electromagnetic motor is turned off. In one example embodiment the elastic unit 307 could be configured in any format at any place along the guiding rail 303.

In one example embodiment, the non-contact scanning process is performed by the first sensor 206a in a cross-artery direction and the distance between the first sensor 206a and the skin surface is controlled to be within 1-2 mm. In one embodiment, the first driving unit 212 will control the movement of the sensor assembly 206 to perform the scanning process of the first sensor 206a. The signal reflected from the skin and received by the first sensor 206a is used as a feedback for controlling the sensor-skin distance. During operation, the intensity of the sensed signal varies with the distance between the sensor 206a and the skin surface, in which the stronger the sensed signal is, the closer the sensor 206a is to the skin, while the weaker the sensed signal is, the farther the sensor 206a is from the skin. In order to eliminate the effect on the measurement accuracy of the sensed signal caused by the varied distance between the sensor 206a and the skin surface, a constant distance between the sensor 206a and the skin is controlled. Moreover, when the sensor 206a is close to the artery, for example, 1 mm-2 mm away from the skin surface, the arterial pulsation information could be detected from the sensed signal. By scanning the skin surface along a predetermined path while keeping a constant distance between the sensor 206a and the skin surface within 1 mm-2 mm, a measuring position range that roughly indicates an artery position is identified according to the analysis of the sensed signal. Once the measuring position range is determined, a position fine-tuning procedure may be performed to determine an accurate location of the artery within the position range for the blood pressure measurement. In one example embodiment, the fine-tuning procedure is carried out by driving the first driving unit 212 and the second driving unit 213. During the position fine-tuning procedure, the second sensor 206b collects a plurality of arterial pulsations under a certain hold-down force from multiple positions within the measuring position range to determine a more accurate measuring position.

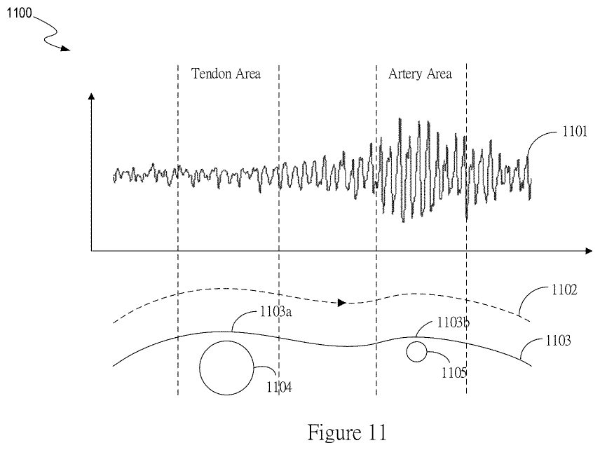

In another example embodiment 1100, as illustrated in FIG. 11, during the non-contact scanning process, the sensed signal 1101 and a scanning trace 1102 of the first sensor 206a will be collected and stored. The scanning trace 1102 of the sensor 206a is used as a representation of a skin contour 1103, as the distance between the sensor 206a and the skin surface is controlled as constant. Once the non-contact scanning process is finished, features of the sensed signal 1101 and the scanning trace 1102 of the sensor 206a can be extracted as inputs for a pre-trained model. In one example embodiment, the pre-trained model is trained and built via machine learning process. The pre-trained model will analyze the sensed signal 1101 and the scanning trace 1102 that represent the skin contour 1103 to predict the artery position for measuring the blood pressure. Usually, the radial artery is under a convex surface of the wrist. However, it may be confused with some protruded front end of tendons. For example, both the skin surfaces 1103a and 1103b above a tendon 1104 and an artery 1105 slightly protrude from the surface. However, the signal with arterial pulsation information sensed within the tendon area is significantly smaller than that within the artery area, as shown in FIG. 11. By training with data including the sensed signal, the scanning trace and the corresponding artery position from a number of people, the pre-trained model is developed and able to precisely predict the artery position, where the wrist surface is protruded and the sensed signal with arterial pulsation information is relatively great, based on the variation of the sensed signal 1101 and the scanning trace 1102. In this one example embodiment, the accuracy of the measuring position determined via the pre-trained model can be increased to within 1 mm from the artery. Therefore, in another embodiment, the position fine-tuning procedure could be omitted and the blood pressure measurement can be directly conducted within the predicted measuring position.

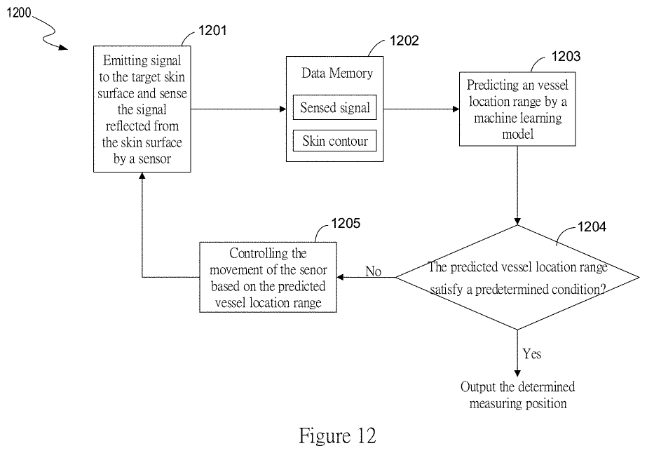

In yet another example embodiment, as illustrated in FIG. 12, during the non-contact scanning process, the sensed signal and the scanning trace of the sensor 206a are continuously collected, stored and processed. After the movement of the sensor 206a to a current scanning position as well as the following measurement in step 1201, attributes of the sensed signal and the scanning position are extracted and stored into a data memory for further processing in step 1202. In one embodiment, after the movement of the sensor 206a to a current scanning position as well as the following measurement in step 1201, the skin contour is determined based on a series of scanning positions. Thereafter, in step 1203, the sensed data that include the data measured during the current and the prior movements, and the scanning trace that is represented by the current and prior scanning positions of the sensor 206a are sent into a pre-trained model. The pre-trained model will then predict an artery position region based on the current and historical sensed data and the sensing trace in step 1203. If the predicted artery position region satisfies a predetermined condition in step 1204, the predicted artery position region will be output as the identified measuring position for further process. Otherwise, the movement of the sensor 206a to a next scanning position will be controlled based on the predicted artery position range in step 1205, and thereafter, the non-contact scanning process will return back to the step 1201. By adopting this scanning method, it may not be needed to scan the whole predetermined range of wrist surface for identifying the artery position as the scanning process will be completed immediately once the artery position is identified by the pre-trained model. Furthermore, by controlling the movement of the sensor 206a based on the predicted artery position range in real-time, it is not needed to move the sensor 206a step by step along the scanning path, but the sensor 206a can be moved in a varied speed to approach the target artery position more quickly. In one example embodiment, speed and efficiency of identifying the artery location will be significantly increased by using this scanning method.

In one example embodiment, the measuring position is predicted via a machine learning process based on the scanned data and the scan path.

In one embodiment, the pre-trained model will predict the artery position and its confidence range, according to which the next movement of the sensor 206a will be controlled. In one example embodiment, the rate of the movement of the sensor 206a depends on a distance between a current position of the sensor 206a and a possible artery range. For example, when the distance between the current position and the possible artery range is greater than a predetermined threshold (i.e. the sensor 206a is far away from the possible artery range), the sensor will move relatively fast as compared to a case where the distance between the current position and the possible artery range is smaller than a predetermined threshold. Therefore, the efficiency of the non-contact scanning process can be increased. The non-contact scanning process will be terminated as long as the confidence range of the predicted artery position meets an accuracy requirement of the measuring position. In one embodiment, the accuracy requirement is that the confidence range of predict position of artery is smaller than 1-2 mm.

In one example embodiment, the pre-trained model predicting artery position is trained and built based on a large amount of prior non-contact scanning data and correspondingly known artery positions. Attributes extracted from the non-contact scanning data are used as model input X and the known artery position is used as model output Y. The model input X and the model output Y are divided into three sets: a training set that includes X_training and Y_training; a validation set that includes X_validation and Y_validation; and a test set that includes X_test and Y_test. The training and validation sets are used for building model and the test set is used for model performance test. The algorithm of pre-trained model can be, but not limited to, support vector machine, linear regression, or artificial neural network.

Referring back to FIG. 3, according to one example embodiment, when the measuring position for the blood pressure measurement on the wrist is identified, the second driving unit 213 (not shown in FIG. 3) will control the sensor assembly 206 to move towards and press the wrist surface 205 at the measuring position for measuring the blood pressure of the user. In one example embodiment, the second driving unit 213 includes a controller 308 for controlling the rotation of a gear (not shown in FIG. 3) or a gear series 310a and 310b to rotate towards or away from the wrist surface 205, so as to enable the sensor assembly 206, which couples to the gear or gear series 310a and 310b, to move towards and press the wrist surface 205, as shown in FIG. 3. In one embodiment, the gear or gear series 310a and 310b are coupled between two guide walls 311 and 312 to prevent the sensor assembly 206 from tilting while pressing the sensor assembly 206 to the wrist surface. In one example embodiment, the guide wall 312 is combined with the controller 308. One of ordinary skill in the art will appreciate that these embodiments are merely examples. For example, the second driving unit 213 could be a mechanical motor such as a pneumatic motor, or an electrical motor such as a step motor or a DC motor, in any configuration with the sensor assembly 206 while satisfying the requirement of driving the sensor assembly 206 to move towards and press the wrist surface for sensing the blood pressure against the wall of the blood vessel. Additionally, the second driving unit 213 could directly or indirectly couples with the sensor assembly 206 for driving the sensor assembly 206 to move towards the wrist surface 205.

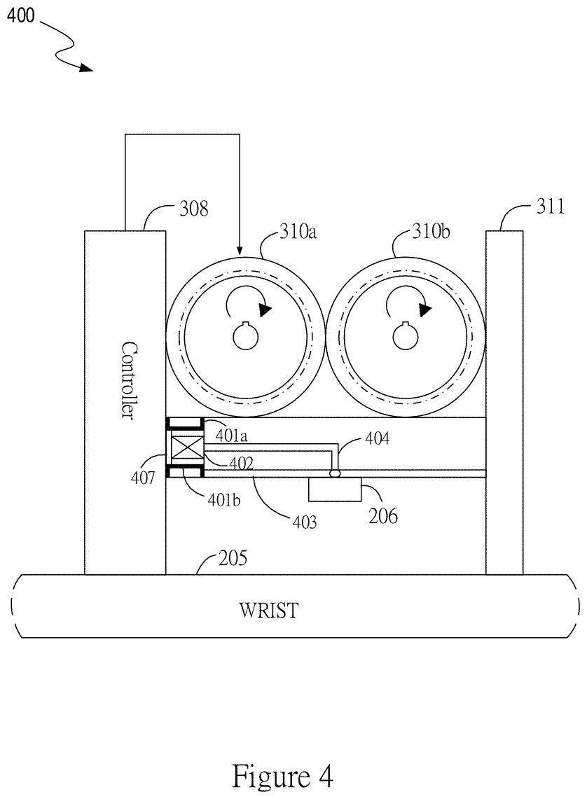

FIG. 4 illustrates another schematic drawing of an electronic device 400 for detecting the blood pressure at the internal side of a user's wrist, according to an example embodiment. FIG. 4 is described in combination with FIGS. 2 and 3.

Elements with the same or similar reference numerals have the same or similar structure/function as thereof in previous figures. In the electronic device 400 of FIG. 4, an electromagnetic motor includes a coil 402 positioned between two magnets 401a and 401b. In one example embodiment, the magnets 401a and 401b are fixed and the coil 402 is movable and connects with a moving unit 404 for driving the moving unit 404 to move along a guiding rail 403 by enabling and adjusting a current flowing through the coil 402. In one embodiment, the moving element 404 is a rolling or sliding element. The moving unit 404 couples with the sensor assembly 206 to bring the sensor assembly 206 to scan the wrist surface 205 in a predetermined path. When the current flows through the coil 402, an electromagnetic force will be generated between the magnets 401a/401b and the coil 402 to enable the coil 402 to move along a direction parallel or substantially parallel to the wrist surface 205. Accordingly, the moving unit 404, which connects to the coil 402, will be driven to move along the guiding rail 403 so as to bring the sensor assembly 206 to scan the wrist surface 205. In an alternative example embodiment, the magnets 401a and 401b are movable and coupled to the moving unit 404 while the coil 402 is fixed. Furthermore, an elastic unit 407 couples with the coil 402 and provides a restoring force to the coil 402. It is understood by one skilled in the art that in addition to the electromagnetic motor as illustrated in FIGS. 3 and 4, the electromagnetic motor could have other alternative configurations to drive the sensor assembly 206 to scan the wrist surface 205. In one example embodiment, the electromagnetic motor is a VCM motor.

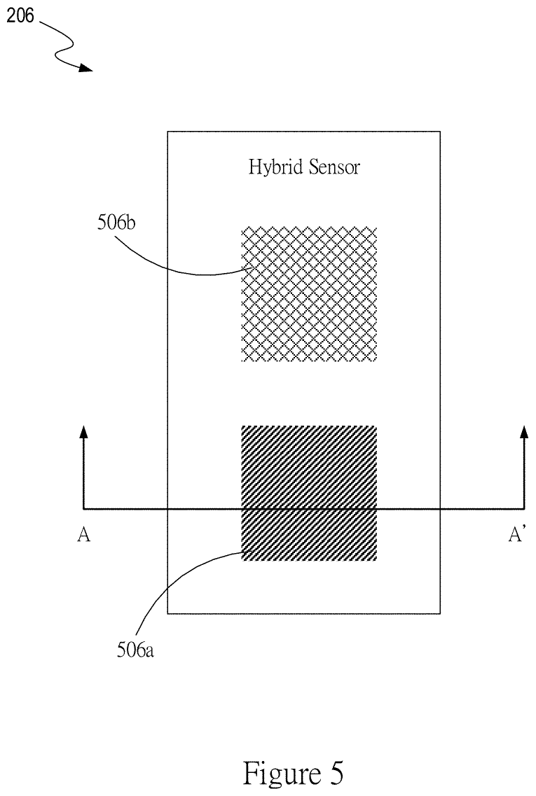

FIG. 5 shows a top view of the sensor assembly 206 used in the electronic device 200 (FIG. 2), in accordance with an example embodiment. FIG. 5 is described in combination with FIG. 2. As shown in FIG. 5, the sensor assembly 206 is a hybrid sensor assembly that includes two sensors 506a and 506b. The first sensor 506a searches a measuring position by scanning the wrist surface 205 without contacting it. The second sensor 506b measures a blood pressure against a blood vessel wall when blood flows through the blood vessel at the measuring position. In one example embodiment, the blood vessel is an artery. The first sensor 506a and the second sensor 506b are disposed on the same side of the sensor assembly 206 that faces the wrist surface 205. A distance between the two sensors 506a and 506b is designed within a predetermined threshold range such that a measurement deviation caused by an offset of the two sensors 506a and 506b is acceptable while the two sensors 506a and 506b are isolated from each other. Furthermore, the first driving unit 212 will adjust a position of the sensor assembly 206 on the wrist surface 205 when the measuring position is identified by the first sensor 506a, so as to locate the second sensor 506b at the measuring position for further process.

During the operation, firstly, the sensor assembly 206 is above the wrist surface 205 and driven to scan the wrist surface 205 along a scanning path to determine a position of a target blood vessel by the first sensor 506a. In one example embodiment, the target blood vessel is a radial artery. When the position of the target blood vessel is identified, the sensor assembly 206 stops moving and stays above the position of the target blood vessel. Then, the sensor assembly 206 is driven to move towards the wrist surface 205 and further press against the wrist surface 205 at the position of the target blood vessel so as to measure the blood pressure by the second sensor 506b.

In one example embodiment, absolute pressure readings can be measured by the second sensor 506b, which is calibrated by a reference force gauge. The blood pressure can be derived or estimated from the measured absolute pressure readings.

In another example embodiment, arterial wall activities can be sensed by the second sensor 506b to generate an arterial pressure pulse waveform, which includes information or attributes of a blood pressure propagation velocity/time along an arterial wall, an arterial pulse reflection velocity/time, and a reflection augmentation index of an arterial pulse, etc. The blood pressure can be derived or estimated from the aforesaid information or attributes extracted from the arterial pressure pulse waveform.

In another example embodiment, blood flow activities can be sensed by the first sensor 506a to generate a blood volume pulse waveform, which includes information or attributes of a blood flow velocity, a blood flow reflection velocity/time, and a reflection augmentation index of the blood flow, etc. In one embodiment, the first sensor 506a emits light toward the wrist surface 205 above the artery and detects the light reflected from the wrist, so as to sense the blood flow activities based on the reflected light that carries the blood information within the blood vessel. The blood pressure can be derived or estimated from the aforesaid information or attributes extracted from the blood volume pulse waveform.

Furthermore, according to an example embodiment, the absolute pressure readings, the information or attributes extracted from the arterial pressure pulse waveform, and/or the information or attributes extracted from the blood volume pulse waveform can be used together to derive or estimate the blood pressure. During the measurements of the absolute pressure readings, the arterial pressure pulse waveform and the blood volume pulse waveform, a hold-down force applied to the sensor assembly 206 for pressing against the skin surface is controlled based on the measured pulse waveforms of the first and second sensors 506a and 506b. In one embodiment, the first sensor 506a is an optical sensor and the second sensor 506b is a pressure sensor.

Moreover, as there are much less blood capillaries under the skin surface of the wrist, it is more difficult to measure blood oxygen saturation via the blood capillaries at the wrist as compared to measuring at a finger. Under such conditions, to measure the blood oxygen saturation via the radial artery is a solution as the radial artery is near the wrist surface with increased blood flow. Unfortunately, at the skin surface above radial artery, the mechanical pulsation is so strong that it will affect the reflected pulsations of red and infra-red light and affect the measurement accuracy of pulse oximetry. In one example embodiment, the sensor assembly 206 integrated with the optical sensor and the pressure sensor can be used to accurately measure the blood oxygen saturation at the radial artery.

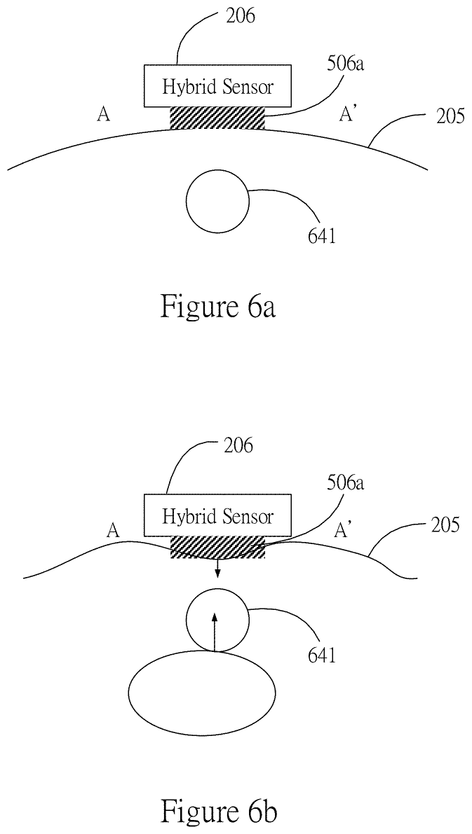

FIGS. 6A and 6B show an operating mechanism of the electronic device 200 with the sensor assembly 206, according to one example embodiment. FIGS. 6A and 6B are described in combination with FIGS. 2 and 5. The cross-sectional view of the sensor assembly 206 in FIGS. 6A and 6B is derived from the line A-A′ of FIG. 5. The sensor assembly 206 is controlled by the first driving unit 212 and second driving unit 213 as described in FIG. 2.

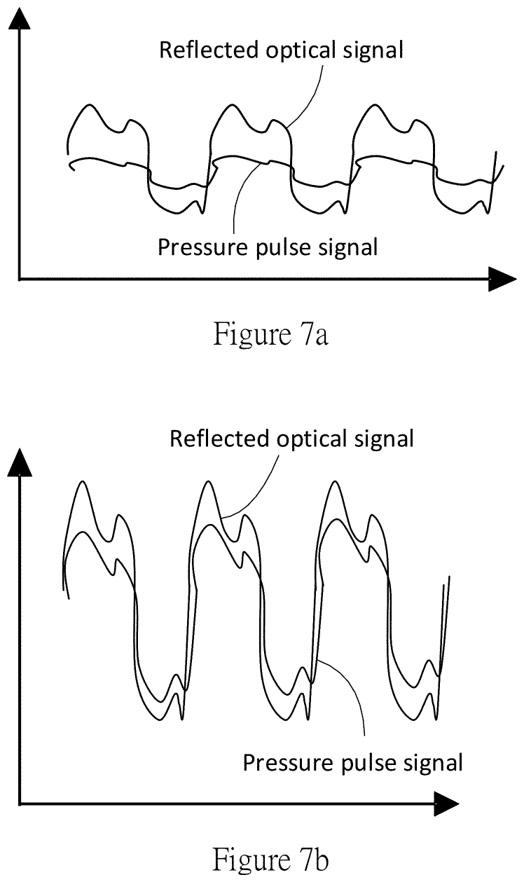

During operation, when the optical sensor 506a identifies the measuring position of the radial artery 641 at the wrist, the sensor assembly 206 will be moved towards the wrist surface 205 at the identified measuring location. Referring to FIG. 6A, when the sensor assembly 206 is driven to move towards the wrist surface 205 at the identified measuring location and touches on the wrist surface 205, an optical signal reflected from the wrist surface 205, so called photoplethysmography (PPG), as shown in FIG. 7A that carries information of blood volume changes can be detected by the optical sensor 506a for the calculation of blood oxygen saturation. In one embodiment, the optical sensor 506a emits light towards the wrist and detects the PPG signal from the wrist. Referring to FIG. 6B, after touching on the wrist surface 205, the sensor assembly 206 will continue to move towards and press against the wrist surface 205 over the location of the radial artery 641 by a predetermined hold-down force until the sensor assembly 206 reaches a predetermined depth for blood pressure measurement.

FIG. 7A shows waveforms of a reflected optical signal and pressure pulse signal detected by the sensor assembly 206 when it is in touch with the wrist surface 205 as illustrated in FIG. 6A, according to one example embodiment. FIG. 7B shows waveforms of the reflected optical signal and pressure pulse signal detected by the sensor assembly 206 when it presses against the wrist surface 205 as illustrated in FIG. 6B, according to one example embodiment. During the operation, the blood oxygen saturation of the user is calculated based on the optical signal reflected from the wrist surface 205 and detected by the sensor assembly 206. More specifically, the blood oxygen saturation is calculated based on a ratio of the AC part to DC part of the optical signal. The AC part of the optical signal is a variable part containing changes caused by both mechanical variation and blood flow. In order to obtain an accurate measurement result of the blood oxygen saturation, it is important to eliminate the effect of the mechanical variation applied to the AC part of the optical signal.

By comparing FIG. 7B with FIG. 7A, although the AC part of optical signal in FIG. 7B is stronger than in FIG. 7A, the increase of AC intensity is mainly induced by mechanical pulsation, as the skin tissue resonance with arterial pulsation is gradually increased when the sensor assembly 206 is pressed towards the radial artery 641, according to an example embodiment. Hence, the measurement accuracy of blood oxygen saturation is affected accordingly. In order to eliminate the influence of mechanical pulsation of the radial artery, it is preferred to avoid deeply pressing the sensor assembly 206 against the radial artery. In another aspect, since the light leakage caused by the gap between the sensor assembly 206 and the wrist surface may also affect the measurement accuracy, the sensor assembly 206 is close to the skin surface to avoid light leakage during the measurement of the blood oxygen saturation. Therefore, on controlling the pressing of the sensor assembly 206 against the wrist surface 205, an optimal contact depth of the sensor assembly 206 upon the wrist surface is determined to balance the impact on the measurement accuracy of the blood oxygen saturation caused by the mechanical pulsation of the radial artery and the light leakage.

Furthermore, as shown in FIGS. 7A and 7B, according to one example embodiment, the pressure pulse signal increases with the increment of a pressed depth of the sensor assembly 206 against the wrist surface 205. In other words, the pressure pulse signal varies with the pressed depth of the sensor assembly 206 against the wrist surface 205. Therefore, the contact depth of the sensor assembly 206 upon the wrist surface could be controlled based on the detected pressure pulse signal to maintain the sensor assembly 206 at the optimal contact depth.

In one example embodiment, when the sensor assembly 206 presses against the wrist surface 205, the pressure pulse between the sensor assembly 206 and the wrist is monitored by the pressure sensor 506b (FIG. 5) to control the hold-down force applied on the sensor assembly 206. To minimize the impact on the measurement accuracy of the blood oxygen saturation caused by the mechanical pulsation and avoid light leakage, the optical sensor 506a will measure the blood oxygen saturation when the pressure pulse is between 0-40 mmHg. In other words, the optimal situation to measure the blood oxygen saturation is when the sensor assembly 206 just touches or slightly press the wrist surface 205. By monitoring the pressure pulse sensed by the pressure sensor 506b, the optimal situation could be identified and maintained by adjusting the hold-down force applied on the sensor assembly 206.

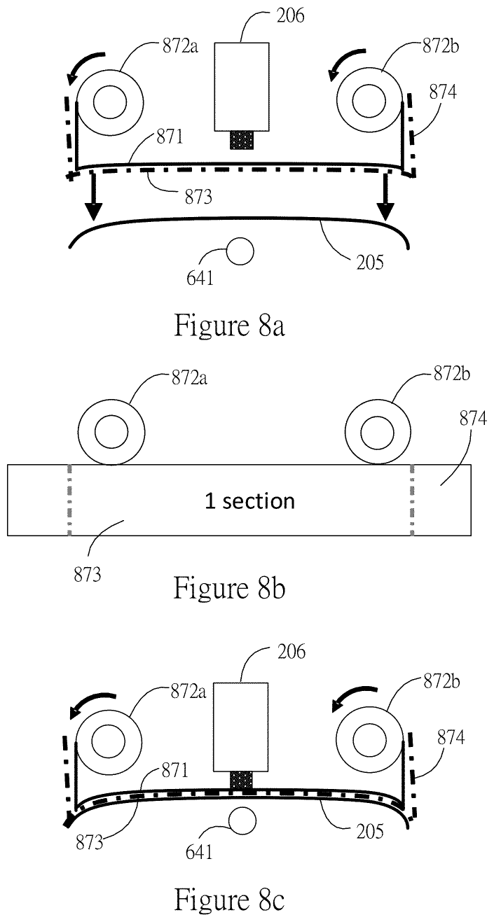

In one example embodiment, to avoid the sensor assembly 206 from contacting the skin surface directly, a membrane is covered on the measuring surface of the sensor assembly 206 to isolate the sensor assembly 206 from the skin surface. FIG. 8A illustrates an exemplary schematic structure of the electronic device 200 with a membrane unit, in accordance with one example embodiment of the presented invention. Referring to FIG. 8A, a section of membrane is added to cover a measuring surface 871 of the electronic device in order to isolate the measuring surface 871 from the user's skin surface 205. To facilitate the user, at least one rolling element that rolls multiple membrane sections one by one is disposed inside the electronic device. In one example embodiment as illustrated in FIG. 8A, the electronic device includes two rolling elements 872a and 872b. In a further example embodiment, the rolling elements 872a and 872b for rolling the membrane sections are controlled manually by the user or automatically by an individual motor controller. In another example embodiment, the rolling elements 872a and 872b are integrated with the first driving unit 212 or the second driving unit 213 (FIG. 2) for rolling the membrane sections. In one example embodiment, the rolling elements 872a and 872b are controlled by the first driving unit 212 and/or the second driving unit 213 for rolling the membrane sections.

In each new measurement, a new membrane section 873 of the membrane unit will be rolled out to cover the measuring surface 871 and a used section 874 will be rolled into the device for withdrawn as specified in FIG. 8A. In one embodiment, the new membrane sections 873 of the membrane unit are stored at one side of the electronic device 200 and the used membrane sections 874 are withdrawn and stored at another side of the electronic device 200.

FIG. 8B shows the new membrane section 873 of the membrane unit from a bottom view, in accordance with one example embodiment. During operation, before each new measurement is made, the used membrane section 874 is rolled back into the electronic device and the new membrane section 873 is consequently rolled out to cover the measuring surface 871. When the electronic device 200 is worn on the user's wrist, the new membrane section 873 will be adhered to the skin surface 205 to prevent the measuring surface 871 of the electronic device 200 from directly contacting the skin of different users while improving the stability of the electronic device 200 as shown in FIG. 8C, so as to avoid cross-contamination between different users.

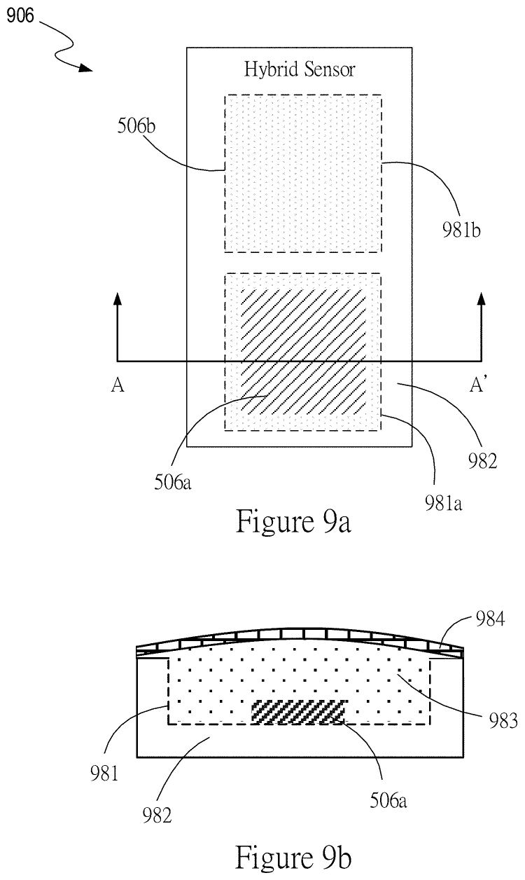

Additionally, FIG. 9A shows a top view of a sensor assembly 906 with a coating layer, according to one example embodiment. FIG. 9B shows a cross-sectional view (from an A-A′ direction of FIG. 9A) of the sensor assembly 906 with the coating layer, in accordance with an example embodiment. FIGS. 9A and 9B are described in combination with FIG. 5. As shown in FIG. 9A, the first sensor 506a and the second sensor 506b are respectively disposed in a first sensor cavity 981a and a second sensor cavity 981b, which are embedded in a substrate 982 of the sensor assembly 906. More specifically, as referring to FIGS. 9A and 9B, the first sensor 506a disposes at a bottom of the first sensor cavity 981a. A transparent material is filled in the first sensor cavity 981a to encapsulate the first sensor 506a so as to protect and prevent the first sensor 506a from directly contacting the outside. In one example embodiment, the transparent material forms an encapsulate layer 983 for the first sensor 506a. Furthermore, a protecting layer 984 is coated on a surface of the sensor assembly 906 in order to not only reduce the friction between the wrist surface 205 and the sensor assembly 906, but also minimize diffusion of moisture into the encapsulate layers of the respective sensors, for example, the encapsulate layer 983 of the first sensor 506a, so as to enhance the reliability of the whole sensor assembly 906. In one example embodiment, the protecting layer 984 is sprayed on to the whole surface of the sensor assembly 906. Configuration of the second sensor 506b within the second sensor cavity 981b could have similar structure as that of the first sensor 506a within the first sensor cavity 981a, as illustrated by FIG. 9b.

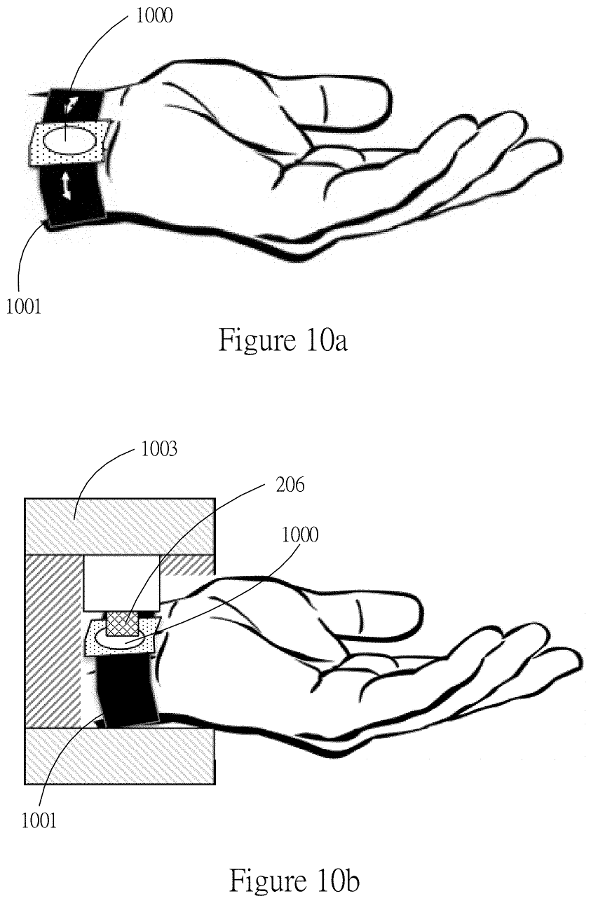

FIG. 10A shows a movable frame 1000 being worn on a user's wrist via a wristband, according to one example embodiment. FIG. 10B shows a portable device that couples to the movable frame 1000 for measuring the health information of the user, according to an alternative example embodiment of the present invention. In the example embodiment shown in FIG. 10A, in order to reduce the burden of the users, a movable frame 1000 is worn on the wrist of a user for receiving the sensor assembly 206, wherein the sensor assembly could be attached to and detached from the movable frame 1000. In one example embodiment, the movable frame 1000 is worn on the wrist via a wristband 1001, as shown in FIG. 10A. In another example embodiment, the movable frame 1000 could be worn on the wrist via gloves, mittens or in other wearable styles. For measuring the health information of the user, as shown in FIG. 10B, a portable device 1003 that includes the sensor assembly 206, the first driving unit 212 and the second driving unit 213 (not shown on FIG. 10B) is disposed on the wrist and the sensor assembly 206 is coupled to the movable frame 1000 manually or automatically. In one example embodiment, the first driving unit 212, the second driving unit 213 and the sensor assembly 206 are detachable from the portable device 1003. In another embodiment, the second driving unit 213 integrates with the sensor assembly 206 and the first driving unit 212 is detachable from the portable device 1003. In yet another embodiment, the first driving unit 212, the second driving unit 213 and the sensor assembly 206 are integrated together with the portable device 1003.

During operation, the first driving unit 212 drives the sensor assembly 206, which couples with the frame 1000, to scan the wrist's skin surface to determine a suitable position for measurement. Then, the first driving unit 212 is detached from the portable device 1003 for load release and the second driving unit 213 will drive the sensor assembly 206 along with the frame 1000 to move towards the wrist skin at the suitable position in order to perform pulse oximetry and blood pressure measurements, in one example embodiment. In another example embodiment, when the suitable position is identified, the second driving unit 213 will start to control the movement of the sensor assembly 206 with the frame 1000 towards the wrist surface without detaching the first driving unit 212 from the portable device 1003. Additionally, when the suitable position is identified, the movable frame 1000 could be locked at the identified position to prevent the displacement/offset of the sensor assembly 206 along the wrist surface during measurement.

After measurement, the portable device 1003 is detached from the wrist to release the load on the user's wrist. In another example embodiment, the sensor assembly 206 is always fixed with the movable frame 1000 to be carried by the user. For measuring the health information, the portable device 1003 with the two driving units 212 and 213 is coupled to the sensor assembly 206 to control the movement of the sensor assembly 206 so as to achieve the measurement as described above.

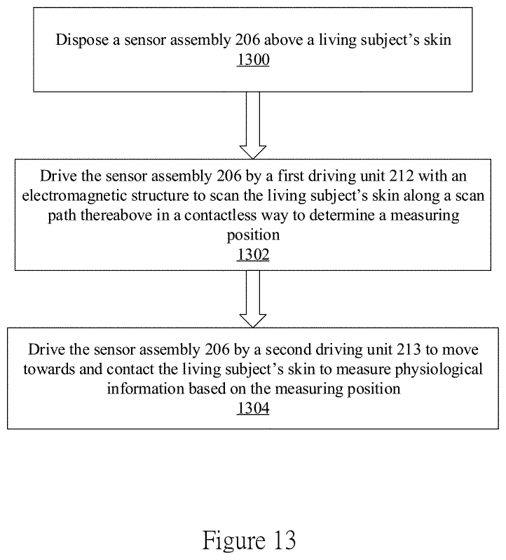

In one example embodiment, FIG. 13 shows a flowchart of an electronic device being applied to a living subject for healthcare measurement. FIG. 13 is described in combination with FIG. 2. A sensor assembly 206 is disposed above a living subject's skin in step 1300. The sensor assembly 206 is driven by a first driving unit 212 with an electromagnetic structure to scan the living subject's skin along a scanning path there above in a non-contact way to determine a measuring position in step 1302. The sensor assembly 206 is driven by a second driving unit 213 to move towards and contact the living subject's skin to measure physiological information based on the measuring position in step 1304.

In one example embodiment, a magnet interacts with a coil of the first driving unit to generate an electromagnetic force for driving the sensor assembly.

In another example embodiment, a moving element is moved along a guiding rail due to the action of electromagnetic force. In yet another embodiment, a friction force is generated between the guiding rail and the moving element during the movement to reduce the shift and improve the stability of the sensor assembly.

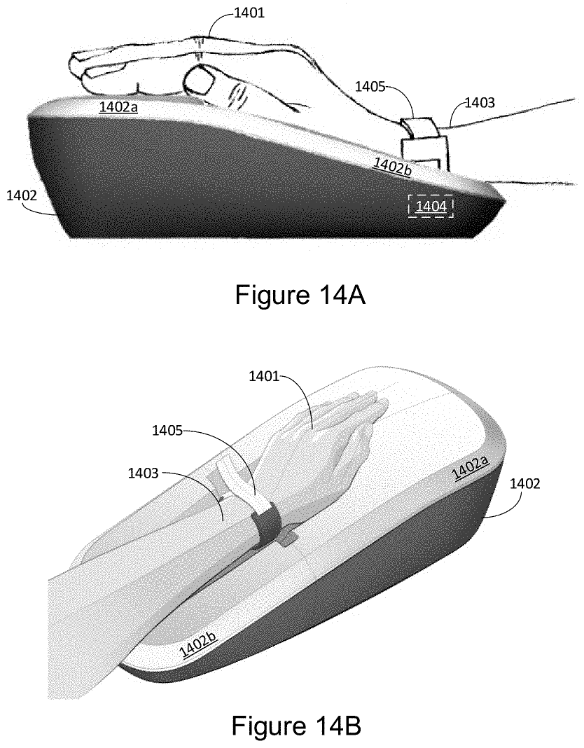

FIGS. 14A and 14B show a schematic drawing of a portable device in operating mode for measuring physiological information of a user, according to one embodiment of the present invention. During the operation, when the user put a hand 1401 onto the device 1402 for measuring the physiological information, the palmar side of the wrist 1403 is towards a sensor 1404 (shown in dot line as configured inside the device) integrated within the portable device 1402, in one embodiment. In a preferred embodiment, the sensor 1404 may be integrated with several sub-sensors, for example, but not limited to, an optical sensor for detecting physiological information of the user in a non-contact mode and a pressure sensor for detecting physiological information of the user in a contact mode. In still a preferred embodiment, when the wrist 1403 is put on the device 1402 as illustrated in FIG. 14, the sensor 1404 is positioned beneath the wrist 1403. Under such configuration, the user will feel more comfortable, relaxed and natural during the measurement. Furthermore, in order to let the palmar surface of the wrist 1403 being fully exposed to the sensor 1404 with enough tension on the wrist, the device 1402 is designed in a high-low trend such that the hand 1401 could be put on a higher front portion 1402a of the device while the wrist 1403 will be located at a lower rear portion 1402b of the device. Under such condition, the palmar skin surface of the wrist 1403 is tensed towards the sensing surface of the device for easing the detection of the vital sign at the wrist 1403.

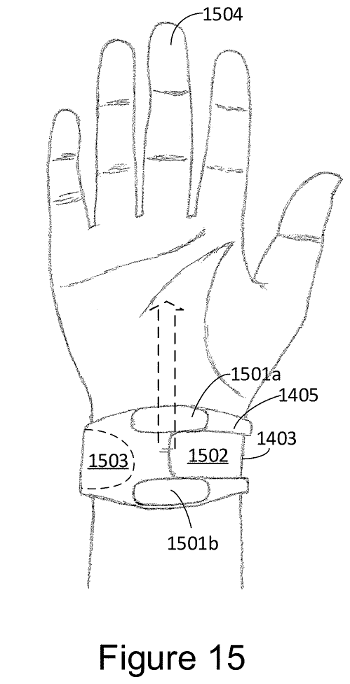

In one embodiment, an additional component could be configured on the user for eliminating the movement of the user during the measurement, especially to limit the movement of the wrist on the device 1402, so as to guarantee the measurement accuracy. In one preferred embodiment, the user will wear a band 1405 on the wrist 1403 before the measurement for fixing the wrist 1403 onto the sensing surface of the device 1402 and prevent the wrist 1403 from shifting, even a small movement, during the measurement. FIG. 15 shows a measuring band wearing on the wrist for the measurement operated by the portable device, according to one embodiment of the present invention. As shown in FIG. 15, there are two ferromagnetic components 1501a and 1501b being symmetrically configured on the two sides of the band 1405, in one embodiment. A sensing opening 1502 is configured between the two ferromagnetic components 1501a and 1501b for defining the sensing area of the wrist 1403. In one embodiment, the sensing opening 1502 is rectangular shaped with one side edge being aligned to the middle of the two ferromagnetic components 1501a and 1501b, and the other side edge nearby the end of the band 1405. As can be understood by one skilled in the art that the shape of the opening could have other applicable structures as long as it satisfies the requirement of defining the sensing area of the wrist 1403. For properly wearing the band 1405 on the wrist 1403 for physiological measurement, the middle of the ferromagnetic components 1501a is aligned with the middle finger 1504 as indicated by a dotted arrow when the band 1405 is worn on the wrist, in a preferred embodiment. By properly wearing the band 1405 on the wrist 1403, the target wrist surface where an artery pulse locates beneath will be exposed to the sensor 1404 through the sensing opening 1502 when the wrist 1403 is put on the device 1402 for measurement. As such, the sensor 1404 is able to detect physiological information at the target wrist surface. Optionally, another opening 1503 could be configured on the band 1405 at the opposite side of the sensing opening 1502, such that when the user wears the band 1405 on the wrist 1403, the styloid process of the ulna could protrude from the sensing opening 1502 such that the user will feel more comfortable.

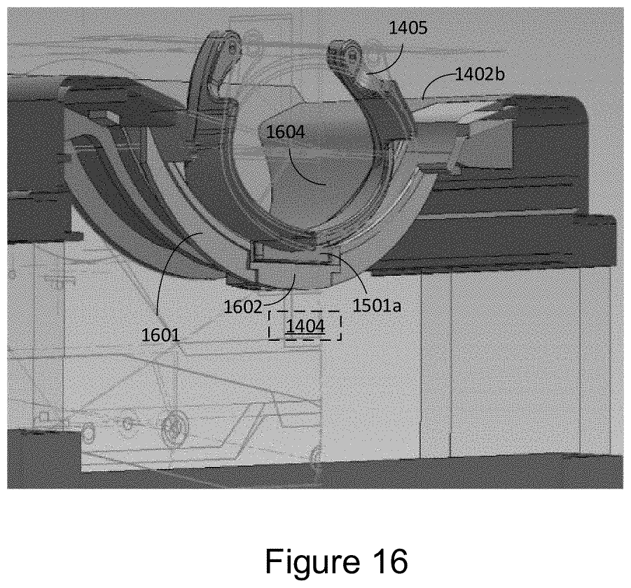

During the operation, when the wrist is put at the lower rear portion 1402b for measurement, the ferromagnetic components 1501a and 1501b will be tightly coupled with the sensing surface of the device 1402 due to a magnetic attraction between the ferromagnetic components 1501a/1501b and the sensing surface. FIG. 16 shows a cross-sectional view of the wristband being magnetically coupled with the device during the operation, according to one embodiment of the present invention. In one embodiment, there is a recess 1604 at the middle of the rear portion 1402b for holding the wrist. An arc-shaped opening 1601 is laterally across the recess surface at a proper position. The sensor 1404 is configured under the opening 1601. Another ferromagnetic component 1602 is configured near by the opening 1601, e.g., near the bottom of the opening 1601 or along the arc-side of the opening 1601, to be coupled with the ferromagnetic components 1501a and 1501b of the wristband 1405 when the user puts the wrist 1403 on the device 1402 for measurement as illustrated in FIGS. 14A and 14B. Under such configuration, the wrist 1403 will be held at the recess 1604 and the band 1405 is stably coupled with the opening 1601 due to the attraction between the ferromagnetic components 1501a/1501b and 1602. Under such condition, the wrist 1403 could be fixed on the device 1402 without unwanted shift during the measurement. The skin surface of the wrist 1403 will expose to the sensor 1404 through the opening 1502 and 1601 which are properly aligned with each other. Thereafter, the sensor 1404 will detect the physiological information of the user at the wrist 1403 through the openings 1502 and 1601. In one embodiment, the sensor 1404 will scan the exposing region of the wrist 1403 defined by the opening 1502 along a predetermined path defined by the opening 1601 to search an optimal position where the artery pulse locates nearby, and then detect the vital signals at the optimal position. Although the embodiments through the whole description mainly describe how to detect an optimal position near the artery pulse and measure the vital signs at the optimal position by the device 1402, it can be also applied to alternative embodiments wherein the device is able to detect an optimal position where another blood vessel pulse is nearby for measuring the corresponding vital signs.

As can be understood by one skilled in the art that, the above embodiment is one example for illustration. In one embodiment, the component 1602 could be a magnet and the components 1501a/1501b could be metal materials that can interact with magnet (e.g., iron), or vice versa. In another embodiment, the components 1602 and 1501a/1501b are both magnets that could be attracted with each other. Moreover, the environmental design surrounding the wristband 1405 and the opening 1601 is not limited to the example as shown in FIG. 16 and could be amended according to different requirements.

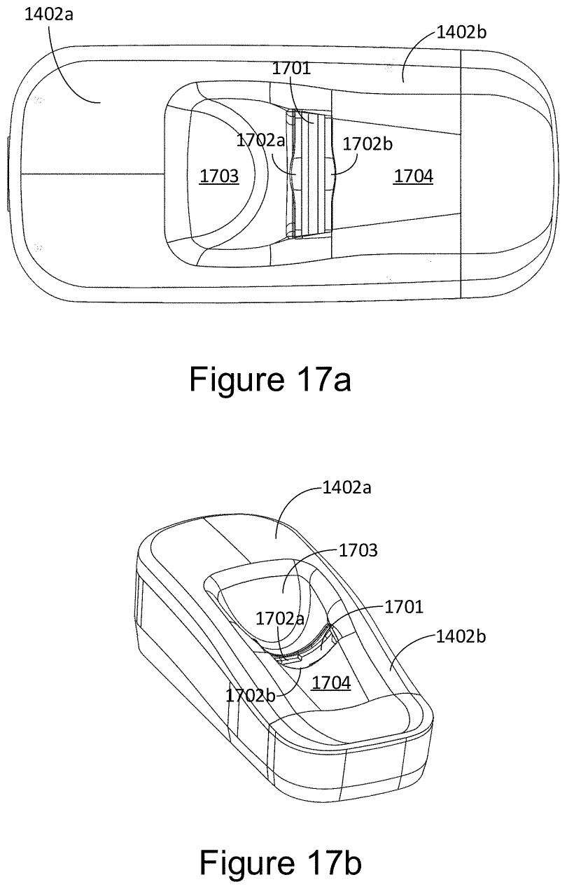

FIGS. 17a and 17b respectively illustrate schematic drawings of a top view and a perspective view of the device 1402, according to one embodiment of the present invention. As shown in FIGS. 17a/17b, a recess 1704 is configured at the middle of the rear position 1402b of the device 1402 for holding the wrist 1403. An arc-shaped opening 1701 is laterally across the recess 1704 while perpendicular to the hand-wrist direction. Two ferromagnetic components 1702a and 1702b are configured at the bottom of the opening 1701. When the user puts the hand on the device for measurement, the wrist 1403 is held by the recess 1704 while the ferromagnetic components 1501a and 1501b of the band 1405 are respectively coupled with the ferromagnetic components 1702a and 1702b of the opening 1701 on the recess 1704. In a preferred embodiment, the configuration of the components 1702a and 1702b are well designed that when the components 1501a and 1501b are coupled with the components 1702a and 1702b, the sensing opening 1502 of the band 1405 is accurately aligned with the opening 1701 of the device 1402 to provide enough measuring space for the sensor 1404 to detect the pulse position on the wrist and measure the vital signs at the pulse position. Furthermore, a slope 1703 exists between the higher front portion 1402a and the lower rear portion 1402b as a buffer between the hand 1401 and the wrist 1403 to enhance the user experience.

In an alternative embodiment, the front portion 1402a of the device 1402 is movable from the main body of the device 1402, in order to fit different sizes of users' hands-wrists. During the operation, when a user wears the band 1405 and prepares to put the hand-wrist onto the device 1402, the user will adjust the position of the front portion 1402a by extending it from or drawing it back to the main body of the device 1402 to find his/her most comfortable position to put the hand-wrist on.

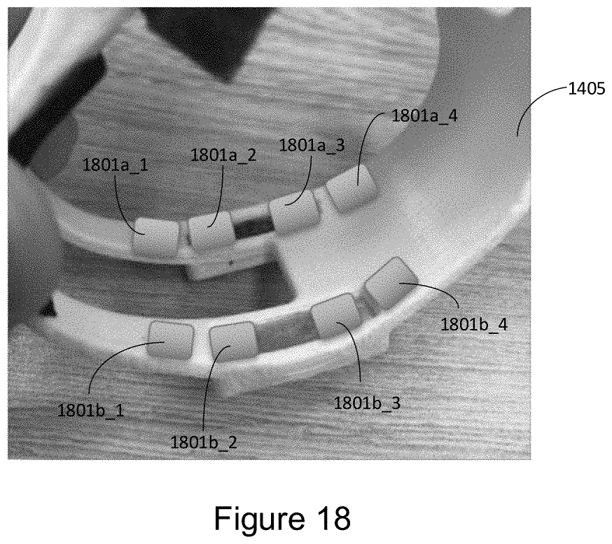

Furthermore, the shape and configuration of the ferromagnetic components 1501a/1501b and 1602 are not limited to the examples shown in FIGS. 15 and 16. In an alternative embodiment, as exemplarily illustrated by FIG. 18, the one-piece ferromagnetic component 1501a could be separated into several blocks, e.g., four blocks 1801a_1, 1801a_2, 1801a_3 and 1801a_4, that are distributed along one side of the band 1405. Similarly, the one-piece ferromagnetic component 1501b could be separated into several blocks, e.g., four blocks 1801b_1, 1801b_2, 1801b_3 and 1801b_4, that are distributed along another side of the band 1405. In a specified embodiment, several blocks, e.g., blocks 1801a_1, 1801a_2, 1801a_3 and 1801a_4, are evenly distributed along one side of the band 1405 and several blocks, e.g., blocks 1801b_1, 1801b_2, 1801b_3 and 1801b_4 are evenly distributed along another side of the band 1405, as exemplarily illustrated in FIG. 18. Under such configuration, enhanced magnetic force could be generated along a wide range of the band sides, and the wrist 1403 with band 1405 will be more tightly and stably coupled with the device 1402. Furthermore, such separated configuration could enable the user to wear the band 1405 more easily as the band 1405 could be smoothly bended. Correspondingly, the configuration of the ferromagnetic components 1602 at the device 1403 will be changed to match the separated configuration of the ferromagnetic components 1501a/1501b. In another embodiment, only one side of the band 1405 is configured with ferromagnetic component, no matter in one-piece or separated blocks, for coupling the wrist 1403 to the device 1402. Accordingly, the configuration of the ferromagnetic component 1602 at the device 1402 will be also changed to match the one-side configuration of the ferromagnetic component at the band 1405.

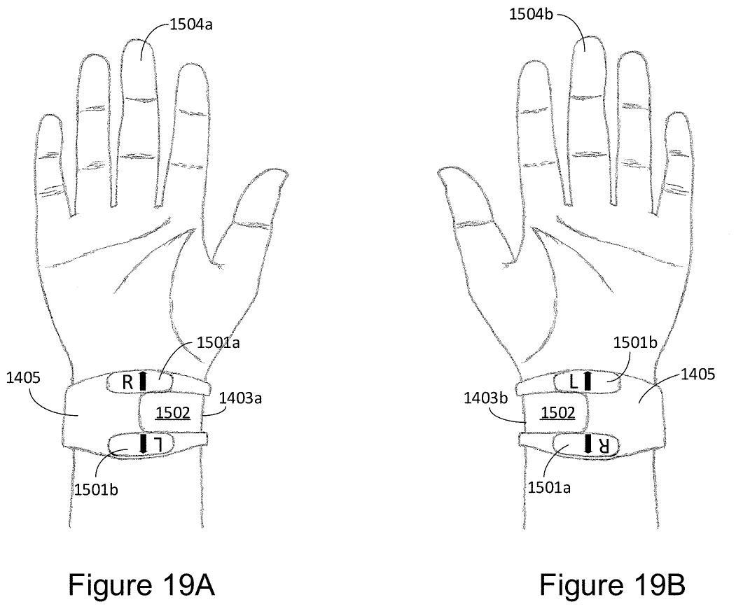

In one embodiment, the user can put either the left/right wrist on the device 1402 for measuring the vital signs, e.g., pulse rate, blood pressure, etc. The band 1405 is also designed to fit for wearing on either wrist. In one embodiment, instruction signs are marked on the band 1405 for helping the user to properly wear the band 1405 on the left or right wrist. As exemplarily illustrated in FIGS. 19A and 19B, instruction signs are marked on the ferromagnetic components 1501a and 1501b of the band 1405. In one embodiment, the instruction signs include a letter sign indicating which wrist (left or right wrist) it refers to, and an arrow sign besides the letter sign indicates the proper wearing manner of the band 1405 on the current wrist to which the corresponding letter sign refers. When the user wears the band 1405 on the right wrist 1403a, the arrow sign, e.g., marked on the ferromagnetic component 1501a, besides the letter sign “R” will point towards the middle finger 1504a of the right hand. Such that, the sensing opening 1502 will cover an area of the right wrist 1403a where the artery pulse locates beneath. In other word, the area where the artery pulse locates beneath will be exposed through the sensing opening 1502, when the band 1405 is properly worn on the right wrist 1403a according to the instruction signs. When the user wears the band 1405 on the left wrist 1403b, the arrow sign, e.g., marked on the ferromagnetic component 1501b, besides the letter sign “L” will be pointed towards the middle finger 1504b of the left wrist 1403b. As such, the sensing opening 1502 will cover an area of the left wrist 1403b where the artery pulse is located beneath.

As can be understood by one skill in the art, the instruction signs could have other patterns and/or could be marked anywhere on the band 1405 as long as they can help the user to properly wear the band, and are not limited to the embodiment illustrated by FIGS. 19A and 19B.

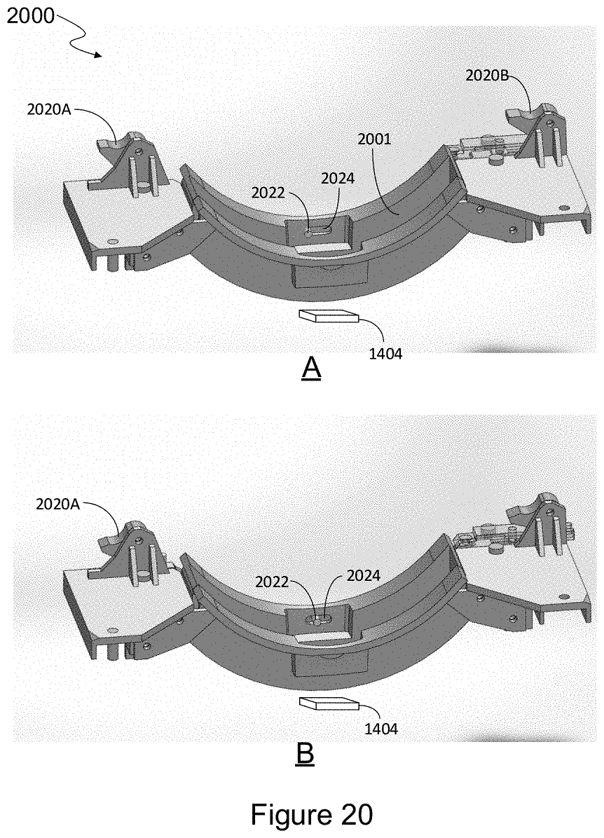

FIG. 20 shows a measurement module 2000 of the device, in accordance with another embodiment of the present invention. In a typical embodiment, the module 2000 is configured on the rear portion of the device, e.g., the rear portion 1402b of the device 1402. When the user puts the wrist onto the device, the wrist is coupled with the module 2000 for measurement. More specifically, the module 2000 comprises an opening 2001 for the sensor 1404 to detect physiological information of the user on the wrist when the wrist is put on the device while coupling with the opening 2001. In one embodiment, the user wears a wristband on the wrist during the measurement. A locking mechanism is configured on at least one side of the opening 2001 for affixing the wristband to the opening 2001. In one example embodiment, a latch unit 2022 configured within a locking rail 2024 is controlled by at least one control unit 2020A. By controlling the control unit 2020A, e.g., to press the control unit 2020A from status A to status B as exemplarily shown in FIG. 20, the latch unit 2022 will move along the locking rail 2024 to lock the wristband. In an alternative embodiment, the module 2000 comprises two control units 2020A and 2020B for controlling the status of the latch unit 2022. As such, when the user puts either one of the wrists (left and right wrists) on the device for measurement, the other hand of the user could press the nearer one of the control units 2020A and 2020B for facilitating the process.

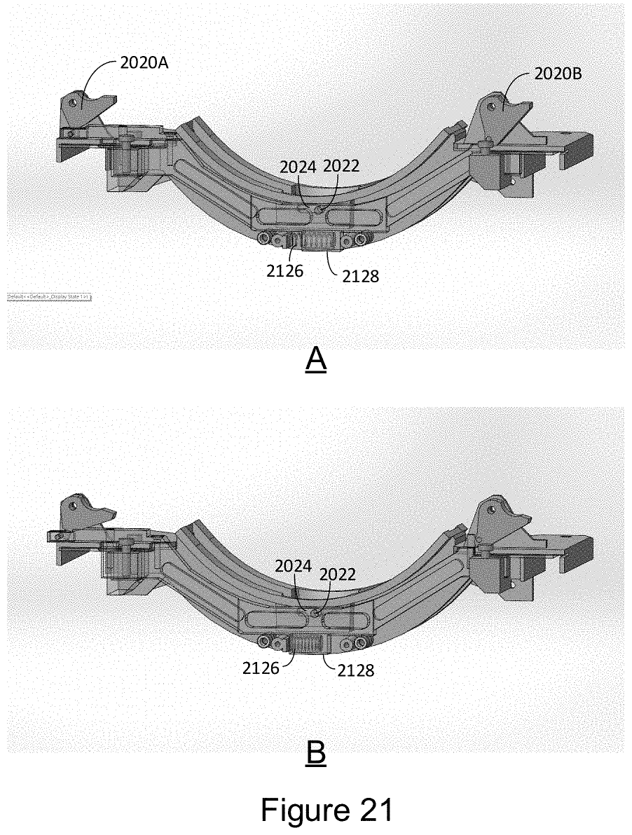

FIG. 21 illustrates a locking mechanism of the device for locking the wristband during the operation, in accordance with another embodiment of the present invention. FIG. 21 will be described in combination with FIG. 20 for easy understanding. As shown in FIG. 21, the latch unit 2022 is able to move along the locking rail 2024. An actuator comprising a spring 2126 and a driving unit 2128 is coupled with the latch unit 2022 for driving the latch unit 2022. More specifically, the driving unit 2128 is coupled with the latch unit 2022 to drive the latch unit 2022 moving along the locking rail 2024. In one embodiment, when the control unit 2020A and/or the control unit 2020B is pressed from stage A to stage B, the driving unit 2128 will be actuated to drive the latch unit 2022 to move along the locking rail 2024. Furthermore, the spring 2126 is further coupled with the driving unit 2128 for providing a restoring force on the driving unit 2128 when the control unit 2020A and/or the control unit 2020B is pressed from stage A to stage B and the driving unit 2128, along with the latch unit 2022, is moved from an original position, e.g., the right side in stage A, to a target position, e.g., the left side in stage B. As shown in stage B, the spring 2126 is distorted due to the movement of the driving unit 2128 so as to provide the restoring force on the driving unit 2128.

After the latch unit 2022 is driven to the target position in stage B, the user will put a wrist with a wristband onto the device and couple the wristband to the opening 2001 of the module 2000. When the control unit 2020A and/or the control unit 2020B is released, the driving unit 2128 will be returned to the original position because of the restoring force. Accordingly, the latch unit 2022 will be also driven back to the original position for locking the wristband. Therefore, the wrist is affixed to the opening 2001 for stable measurement. After then, the sensor 1404 will begin to sense the physiological information of user at the sensing area defined by the wristband through the opening 2001.

The mechanism for eliminating the movement of the wrist on the device is not limited to the embodiments as elaborated above. Other solutions could be also applied once satisfied the requirement, e.g., to use an inflatable cuff behind the wrist for eliminating the wrist's movement, or to couple the arm of the user with a fixing component to control the arm's movement during the measurement.

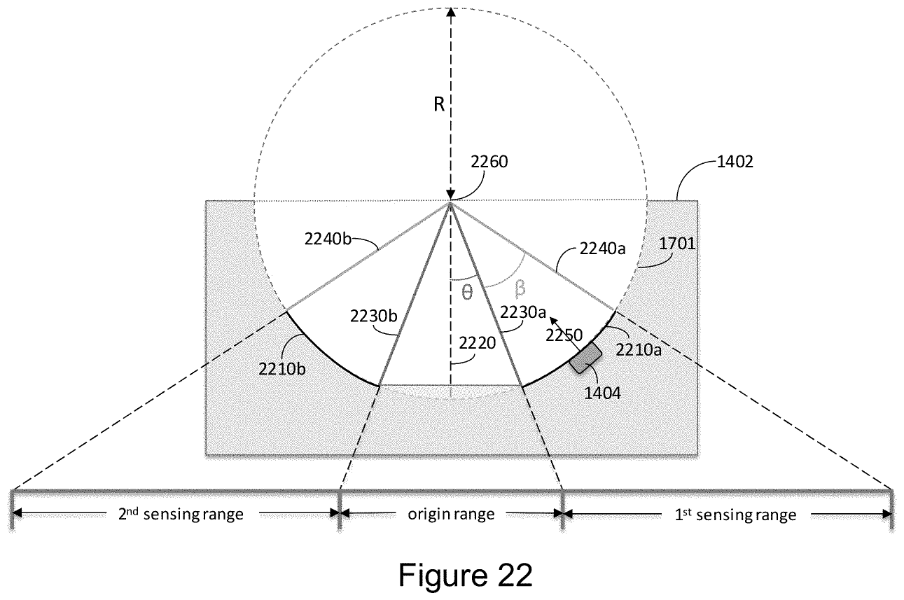

FIG. 22 illustrates a schematic drawing of the operating mode of the sensor 1404 for detecting the vital signs on the user's wrist, according to one embodiment of the presented invention. For illustration purposes, the sectional view of the device 1402 is set as seen from the rear portion 1402b to the front portion 1402a. At an initial phase, the sensor 1404 will be stopped at an origin position 2220, e.g., at the middle bottom of the opening 1701. Optionally, the sensor 1404 could be stopped within an origin range surrounding the origin position 2220 as indicated in FIG. 22. When the user puts the wrist on the device 1402 for detecting vital signs at the wrist, the user will firstly set the initial sensing status of the sensor 1404 by moving the sensor 1404 to a first initial sensing position 2230a or to a second initial sensing position 2230b as indicated in FIG. 22 according the which wrist (left or right) is put on the device, in one embodiment. In one embodiment, the sensor 1404 will be moved to the first initial sensing position 2230a if the user put the left wrist onto the device 1402, or be moved to the second initial sensing position 2230b if the user puts the right wrist onto the device 1402, or vice versa. In one embodiment, the user could set the initial sensing status of the sensor 1404 by pressing a control button configured on the device 1402, or by rotating a knob or through other mechanical manner. In an alternative embodiment, the user would move the sensor 1404 to the first or second initial sensing position by hand. In still an alternative embodiment, the user would set the initial sensing position of the sensor 1404 by wireless control.

After the sensor 1404 being moved to an initial sensing position (here take the first initial sensing position as example for illustration below), the sensor 1404 begins to scan the target skin area of the corresponding wrist to detect an optimum position where the artery pulse locates beneath. In one embodiment, the sensor 1404 is configured to move along a predetermined path above the skin. As illustrated in FIG. 22, the sensor 1404 scans the wrist through the opening 1701 by moving along a predetermined path, e.g., along an arcuate scanning path 2210a within a 1st sensing range if a left wrist is put on the device 1402 or along an arcuate scanning path 2210b within a 2nd sensing range if a right wrist is put on the device 1402, or vice versa. In one embodiment, as indicated in FIG. 22, the sensor 1404 is rotated around a predetermined center 2260 within the 1st or 2nd sensing range to scan the wrist surface along a predetermined path, e.g., the arcuate scanning path 2210a and/or 2210b. The rotation radius R of the sensor 1404 is pre-set within a range of 40-60 mm. The initial sensing angle θ between the origin position 2220 and the 1st/2nd initial sensing position relative to the center 2260 is pre-set within a range of 10-20 degree. The largest rotation angle β of the sensor 1404 within the 1st or 2nd sensing range is pre-set within a range of 20-40 degree. The effective 1st or 2nd sensing range of the sensor 1404 on the wrist surface will be within a range of 10-30 mm.

However, As can be understood by one skilled in the art, the embodiment in FIG. 22 as described above is for exemplary illustration. The origin position 2220, the first initial sensing position 2230a, the first sensing range, the second initial sensing position 2230b and/or the second sensing range are not limited to the above embodiment and can be changed to other workable positions if needed. For example, the sensor 1404 could move in either a forward or a backward direction within the 1st or 2nd sensing range to scan the wrist surface, in an optional embodiment. If the first/second initial sensing positions of the sensor 1404 are set to an position 2240a/2240b as shown in FIG. 22, the sensor 1404 will further move in a backward direction along the predetermined path 2210a/2210b within the corresponding sensing range, or even move back and forth for several times to find the target position more accurately. In an alternative embodiment, the initial sensing position is set as the origin position 2220. The sensor 1404 starts to scan the wrist surface from the origin position 2220 and swept along the opening 1701 to find the target position. Moreover, the rotation radius R, the initial sensing angle θ, the largest rotation angle β, and the effective sensing range could be adjusted according to different requirements or conditions.

Furthermore, during the scanning operation, the sensor 1404 will be operable to scan the skin surface of the wrist by emitting light to the skin surface and detecting light returned from the skin surface, and determine an optimal position, where the artery pulse is the strongest, based on the detected light, in one embodiment. In alternative embodiments, the sensor 1404 could non-contactingly (i.e., without physically contacting) scan the skin surface by emitting and detecting other wireless signals, e.g., MRI or X-ray signal. In still an alternative embodiment, the sensor 1404 scans the skin surface in a contacting manner by emitting and detecting the ultrasound signal or other mechanical wave signal. Thereafter, the sensor 1404 will measure the user's vital signs at the determined optimal position of the wrist. In one embodiment, the sensor 1404 will press the skin surface of the wrist at the determined optimal position and measure the pressure signal against the wrist to detect vital signs, e.g., blood pressure, pulse rate, and/or blood oxygen saturation value, etc. In a preferred embodiment as illustrated in FIG. 22, when the sensor 1404 determines the pulse location of the wrist, the sensor 1404 will then be controlled to move substantially toward the predetermined center 2260, as indicated by an arrow 2250, at the determined pulse location to contact and further press the skin surface of the wrist. Of cause, the direction of the arrow 2250 is not limited to the example illustrated in FIG. 22 and could be properly adjusted according to different requirements. In alternative embodiment, the sensor may non-contactingly (i.e., without physically contacting) detect the vital signs at the optimal position by emitting wireless signal, e.g., optical signal, to the wrist surface at the optimal position and detecting the returned wireless signal reflected from the wrist. In other words, the sensor 1404 could also detect the vial signs of the user in an optical manner.

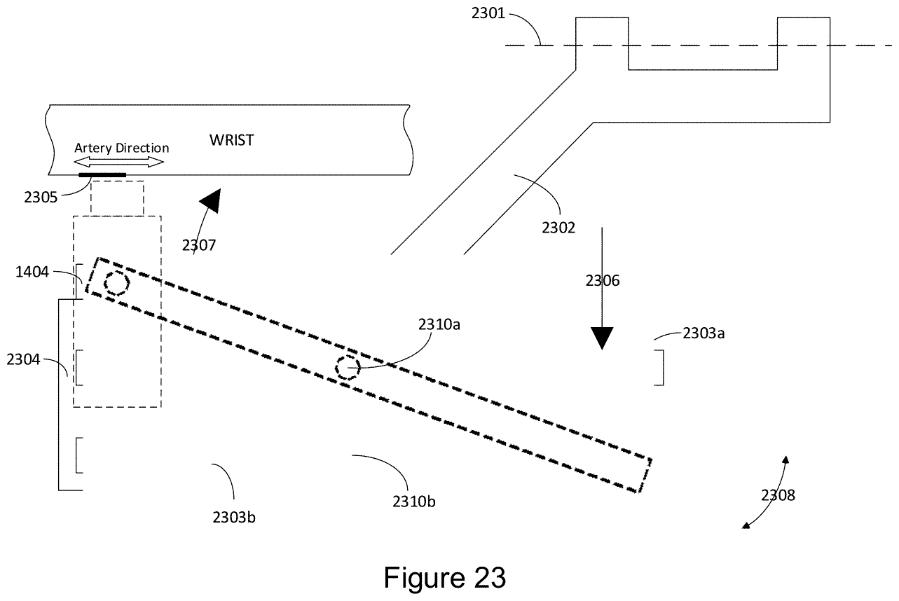

FIG. 23 illustrates a schematic drawing of a mechanical structure of the sensor 1404, in accordance to an exemplary embodiment. As shown in FIG. 23, the sensor 1404 is supported by a moving platform 2304. Two leverage elements 2303a and 2303b are mechanically coupled between the moving platform 2304 and a main cantilever 2302. During the operation, the cantilever 2302 is operable to rotate around an axis 2301 such that the sensor 1404 is brought to move along a predetermined arc path whose direction is substantially perpendicular to the artery direction of the wrist, for example, the arc path within the first/second sensing range as shown in FIG. 22, to scan the wrist surface for detecting the artery pulse position. In one embodiment, the rotation of the cantilever 2302 along the axis 2301 is controlled by a step motor with high control accuracy, e.g., the smallest moving distance of the sensor 1404 driven by the cantilever 2302 is controlled within 0.1 mm.

When the artery pulse position 2305 is determined after scanning, the sensor 1404 will be controlled to move towards the wrist to contact and further press (optional) the wrist surface at the determined position 2305 for vital sign measurement. In one embodiment, the leverage elements 2303a and 2303b are able to revolve on respective coupling elements 2310a and 2310b between the leverage elements 2303a/2303b and the main cantilever 2302, as indicated by an arrow 2308. Therefore, when the leverage element 2303a is pressed in a direction as indicated by an arrow 2306, the leverage element 2303a and 2303b will revolve on the coupling elements 2310a and 2310b, so as to drive the moving platform 2304 along with the sensor 1404 to move towards the wrist in a direction as indicated by an arrow 2307 whose direction is substantially reverse to the direction of the arrow 2306. The arrows presented here roughly shows a moving direction of the sensor 1404 and the real moving direction is not limited to the direction indicated by the arrow 2307. Furthermore, dashed lines presented on the FIG. 23 could clearly demonstrate the sensor's moving condition towards the wrist. As can be seen from the dashed lines and the arrow 2307, during the movement towards the wrist, the sensor 1404 moves in a slightly sloppy direction and the final touch position of the sensor 1404 on the wrist will be slightly deviated from the determined position 2305. However, such deviation will not affect the measurement accuracy since the deviation is negligible within a tolerant range along the artery direction.

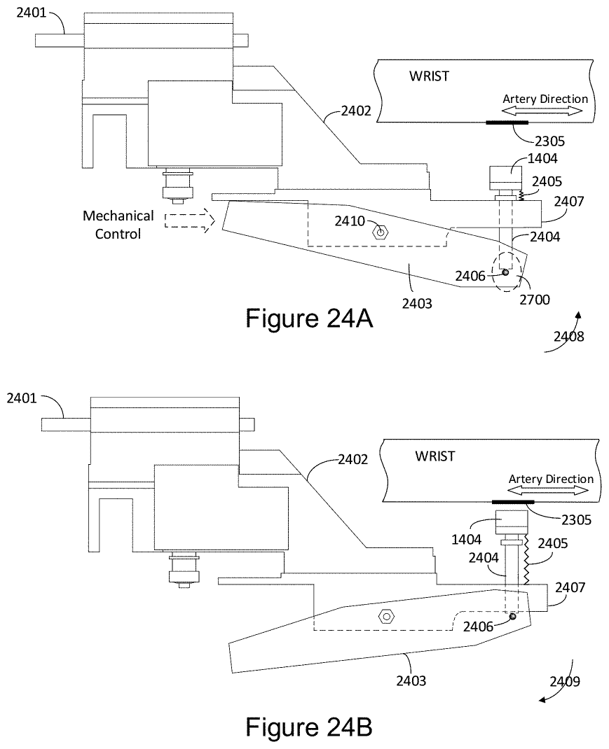

FIGS. 24A-B illustrate a schematic drawing of another mechanical structure of the sensor 1404, according to another exemplary embodiment. As shown in FIG. 24A, the sensor 1404 is configured on a platform 2407 and supported by a supporting element 2404 which penetrates through the platform 2407 via a through hole. The supporting element 2404 could freely move through the hole to drive the sensor 1404 to move away or towards the platform 2407. Furthermore, a leverage unit 2403 is coupled with the platform 2407 via a connecting element 2410, e.g., a screw, and is able to revolve on the connecting element 2410. A resisting element 2406 is configured within the leverage unit 2403, e.g., a bar being coupled between two sides of the leverage unit 2403. The supporting element 2404 is aligned with the resisting element 2406. When the leverage unit 2403 rotates around the platform 2407 in a direction indicated by an arrow 2408, the resisting element 2406 will resist the supporting element 2404 accordingly to lift the supporting element 2404 through the hole of the platform 2407 such that the sensor 1404 will be driven to move away from the platform 2407 while towards the wrist, as illustrated by FIG. 24B.

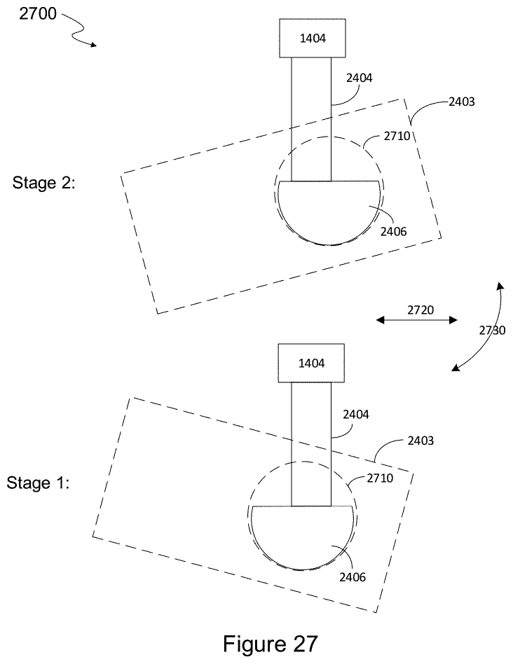

An example showing a detailed mechanical structure among the leverage unit 2403, the resisting element 2406 and the supporting element 2404, as indicated by a dashed ellipse 2700 in FIG. 24A, is illustrated in FIG. 27. As shown in FIG. 27, the resisting element 2406 has a quasi-semicircle or over-semicircle structure that at least a top surface thereof which is flat and loosely coupled with the supporting element 2404, and at least a lateral-side or bottom surface which is arcuate and coupled with a hole 2710 of the leverage unit 2403. The leverage unit 2403 with the hole 2710 are presented by dotted lines, and their real shape could be changed without being limited to the example herein. When the leverage unit 2403 rotates relative to the platform 2407 in the direction as indicated by the arrow 2730, e.g., rotates from stage 1 to stage 2, the resisting element 2406 will roll inside the hole 2710 due to the arcuate lateral-side or bottom surface, so as to keep the top flat surface always horizontal. During the rotation of the leverage unit 2403 from stage 1 to stage 2, the resisting element 2406 will move upwardly and forwardly simultaneously. Since the top flat surface of the resisting element 2406 is kept horizontal, the supporting element 2404 is able to slightly move along the flat surface of the resisting element 2406, as indicated by the arrow 2720, from stage 1 to stage 2. As such, the supporting element 2404 with the sensor 1404 will not move forwardly with the resisting element 2406 during the process from stage 1 to stage 2, which may prevent the sensor 1404 from deviating from the determined optimal position.

In one embodiment, a spring element 2405 is coupled between the sensor 1404 and the platform 2407 to provide a restoring force to the sensor 1404 when the sensor 1404 is moved away from the platform 2407 in FIG. 24B. When the leverage unit 2403 returns to the initial position as indicted by an arrow 2409 in FIG. 24B and the resisting element 2406 does no longer resist against the rod 2404, the sensor 1404 will be pulled back to the platform 2407 by the restoring force of the spring element 2405.

As can be understood by one skilled in the art that the mechanical design among the leverage unit 2403, the resisting element 2406 and the supporting element 2404 are not limited to the above embodiment and can have alternative structures as long as it satisfies the requirement of driving the supporting element 2404 with the sensor 1404 to move towards the wrist without rotating and shifting. For example, in an alternative embodiment, the supporting element 2404 is combined with the resisting element 2406. When the leverage unit 2403 rotates from stage 1 to stage 2, an additional mechanical element will be used to avoid the shift of the resisting element 2406 and the supporting element 2404 along the wrist direction.

Furthermore, the platform 2407 is coupled with a cantilever 2402 which is operable to revolve around a pivot 2401. In one embodiment, the revolution of the cantilever 2402 around the pivot 2401 is controlled by a motor, e.g., the step motor, with high control accuracy, e.g., the smallest moving distance of the sensor 1404 driven by the cantilever 2402 is controlled within 0.1 mm. During the operation, when the cantilever 2402 is driven to revolve around the pivot 2401, the platform 2407 will correspondingly swing beneath the wrist to take the sensor 1404 to move along a predetermined arc path whose direction is substantially perpendicular to the artery direction of the wrist to scan the wrist surface for detecting the artery pulse position. When the artery pulse position 2305 is determined, the sensor 1404 will be then driven to move towards the wrist until contact and press (optional) the wrist skin at the determined position for further measurement, according to the mechanical method as described above.

As can be understood by one skilled in the art, the mechanical design of the sensor 1404 shown in FIGS. 23, 24A and 24B are for exemplary illustration only and the sensor 1404 could have alternative mechanical structures while satisfying the function requirements of the measurement as described above, and not limited to the only embodiments of FIGS. 23, 24A and 24B. Optionally, the cantilever 2302 is able to move along the axis 2301 as controlled by another motor. Under such configuration, the sensor 1404 could be driven to move along the artery of the wrist during the operation to compensate for the deviation from the determined position 2305 when the sensor 1404 is moving towards the wrist. Moreover, by driving the sensor 1404 to move in three directions including along the artery of the wrist; across the artery of the wrist; and toward the surface of the wrist, the sensor 1404 could move more freely to sense physiological information at multiple positions with different pressing force in order to fine-tune the determined position 2305 and achieve more accurate measurement.

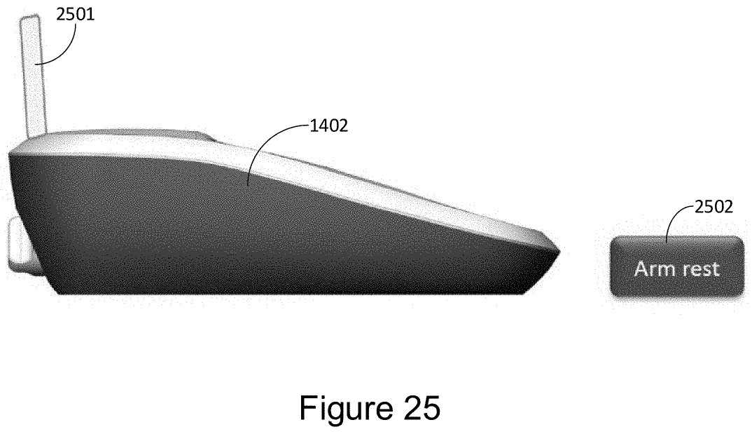

In an optional embodiment, peripheral components could be added for enhancing the user experience and device performance. FIG. 25 shows a schematic drawing of a portable device with peripheral components for measuring physiological information of a user, according to one embodiment of the present invention. FIG. 25 will be described in combination with FIGS. 14A and 14B. As shown in FIG. 25, a display unit 2501 is added in front of the device 1402 for displaying the measurement result, as well as other instructions to the user. The display angle of the display unit 2501 could be adjusted for satisfying different users' requirements. Furthermore, an arm rest component 2502 is added at the back of the device 1402 for resting the user's arm when the user put the wrist on the device 1402. As can be understood by one skilled in the art, the configuration of the display unit 2501 and the arm rest component 2502 could be changed to other formats without limiting to the above embodiment, as long as it can satisfy the subject function. For example, the display unit 2501 could be integrated with the device 1402 and configured upon the top surface of the device 1402. Alternatively, the display unit 2501 could be separated from the device 1402 and only be attached to the device when necessary.

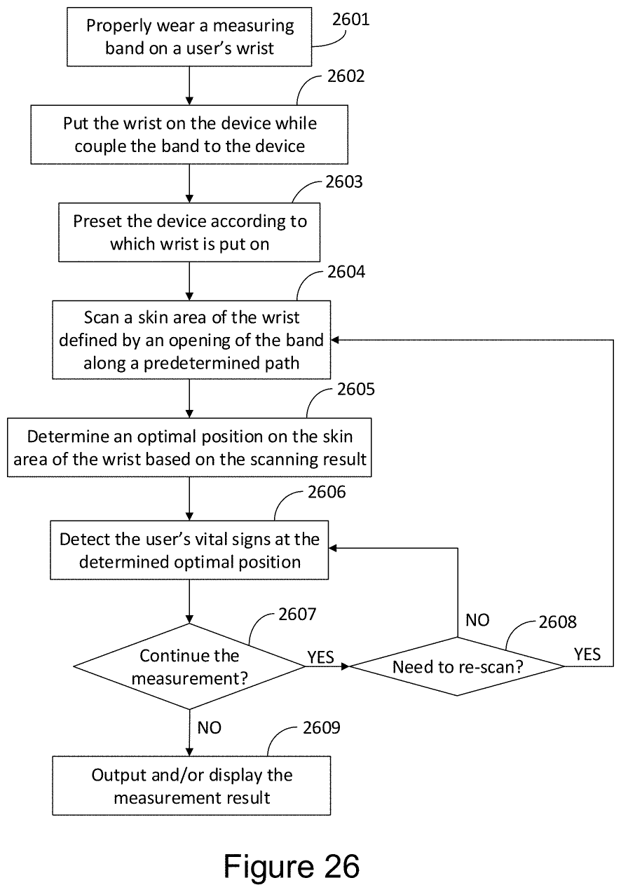

FIG. 26 shows an operation flowchart of a portable device for measuring physiological information of a user, according to one embodiment of the present invention. FIG. 26 will be described in combination with FIGS. 14A and 14B,

FIG. 17A-B, and FIG. 19A-B for easy understanding. As shown in FIG. 26, firstly the user will wear a measuring band, e.g., the band 1405 illustrated in FIGS. 14-19, on the wrist 1403 in step 2601. In one embodiment, when the user properly wears the band 1405 on the wrist 1403, the middle of the ferromagnetic components 1501a is aligned with the middle finger as indicated by the dotted arrow as illustrated in FIG. 15. In a more specific embodiment, the user will wear the band 1405 according to the instruction signs of the band 1405 as exemplarily illustrated in FIGS. 19A and 19B. In FIG. 19A, when the user wears the band 1405 on the right wrist 1403a, the arrow sign besides the letter sign “R” will point to the middle finger of the right wrist 1403a. Such that the sensing opening 1502 will be positioned at the area of the right wrist 1403a where the artery pulse locates beneath. In FIG. 19B, when the user wears the band 1405 on the left wrist 1403b, the arrow sign besides the letter sign “L” will point to the middle finger of the left wrist 1403b. Such that the sensing opening 1502 will be positioned at the area of the left wrist 1403b where the artery pulse locates beneath.

In step 2602, the user puts his/her wrist 1403 on the device 1402 while coupling the band 1405 to the device 1402. During the operation, the user puts the wrist 1403 with band 1405 at the lower portion 1402b of the device 1402, wherein the wrist 1403 is held by the recess 1704 and the band 1405 is coupled with the opening 1701, as exemplarily illustrated in FIG. 17A-B. Additionally, the user puts the hand 1401 on the front portion 1402a of the device 1402 in a comfortable status. In step 2603, the device 1402 is preset according to which wrist (let or right) is put on device 1402. In one embodiment as illustrated in FIG. 22, if the left wrist is put on the device 1402, the sensor 1404 is configured to the 1st initial sensing position within the first sensing range. If the right wrist is put on the device 1402, the sensor 1404 is configured to the 2nd initial sensing position within the second sensing range. As can be understood by one skilled in the art that, the above embodiment is for illustration and the preset rule could be changed to other manner as long as it satisfies the requirement of being applicable to either of the left and right wrists. In step 2604, the sensor 1404 starts to scan a skin area of the wrist 1403 defined by the opening 1502 of the band 1405 along a predetermined path. In one embodiment, the sensor 1404 scans the skin surface of the wrist 1403 by emitting optical signal to the skin surface and detecting the optical signal reflected from the skin surface.

In step 2605, based on the scanning result, the sensor 1404 analyzes the detected optical signal and determines an optimal position on the skin surface of the wrist 1403 for further measurement. In step 2606, the sensor 1404 measures the user's vital signs at the determined optimal position. In one embodiment, the sensor 1404 is controlled to firstly move towards the wrist 1403 until contact and press against the wrist skin surface at the determined optimal position. In a preferred embodiment, the sensor 1404 is controlled by an optimal hold-down force to press on the wrist surface for fine-tuning measurement location and measuring the pressure signal against the wall of the artery under the wrist surface. Based on the measured pressure signal, the sensor 1404 could determine the vital signs of the user, e.g., the blood pressure, the pulse rate, the pulse oximetry, etc. In an alternative embodiment, the sensor 1404 could detect the user's vital signs at the optimal position by optical means. More specifically, the sensor 1404 will emit optical signal to the wrist surface at the optimal position and detect the optical signal passing through the wrist surface and reflected by the artery under the wrist surface. Based on the detected optical signal, the sensor 1404 could determine the vital signs of the user, e.g., the blood pressure, the pulse rate, the pulse oximetry, etc.

In step 2607, if the measurement is determined to continue, then the operation goes to step 2608 to determine whether a re-scan process is needed. If yes, the operation will return to the step 2604 for a next round of scan and measurement process. If not, the operation will return to the step 2606 for a next round of measurement process. In step 2607, if the measurement is determined to stop, then the operation goes to step 2609. In step 2609, the measurement result is output and/or displayed to the user for further process.

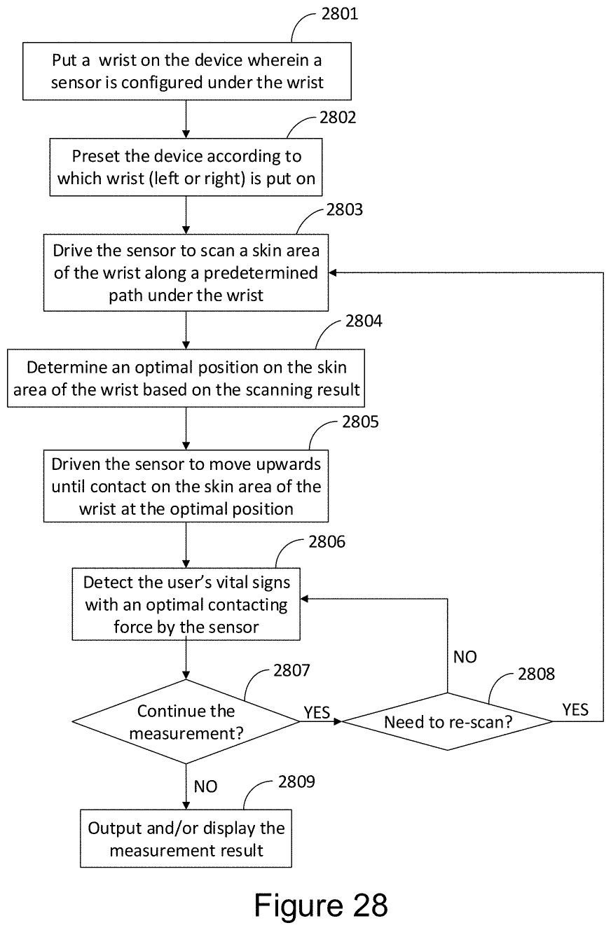

FIG. 28 shows an operation flowchart of a portable device for measuring physiological information of a user, according to another embodiment of the present invention. FIG. 28 will be described in combination with FIGS. 14A and 14B, FIGS. 17A-B, and FIGS. 19A-B for easy understanding. Steps of FIG. 28 which have similar embodiments as the steps of FIG. 26 will be briefly described. As shown in FIG. 28, in step 2801, the user puts a wrist on the device wherein a sensor 1404 is configured under the wrist. In one embodiment, the skin surface of the wrist will be exposed to the sensor 1404 via an opening of the device, e.g., the opening 1701 of the device in FIGS. 17a-17b. In an optional embodiment, the wrist will be properly coupled with the device with additional component for restricting the movement of the wrist. In step 2802, the device is preset according to which wrist (let or right) is put on device 1402. In alternative embodiment, this step could be omitted. In step 2803, the sensor 1404 is driven to scan the skin area of the wrist along a predetermined path under the wrist. In one embodiment, the sensor 1404 is driven to swing under the wrist to scan the skin area of the wrist through the opening 1701 of the device.

In step 2804, an optimal position is determined by the sensor 1404 on the skin area of the wrist based on the scanning result. In step 2805, the sensor is driven to move upwards until contact on the skin area of the wrist at the optimal position. In step 2806, the sensor 1404 will detect the user's vital signs with an optimal contacting force on the skin surface of the wrist. In one embodiment, the sensor 1404 will press on the skin surface of the wrist while adjusting the pressing force to find the optimal contacting force. In step 2807, if the measurement is determined to continue, then the operation goes to step 2808 to determine whether a re-scan process is needed. If yes, the operation will return to the step 2803 for a next round of scan and measurement process. If not, the operation will return to the step 2806 for a next round of measurement process. In step 2807, if the measurement is determined to stop, then the operation goes to step 2809. In step 2809, the measurement result is output and/or displayed to the user for further process.

While the foregoing description and drawings represent example embodiments of the present invention, it will be understood that various additions, modifications and substitutions may be made therein without departing from the spirit and scope of the principles of the present invention as defined in the accompanying claims. One skilled in the art will appreciate that the invention may be used with many modifications of form, structure, arrangement, proportions, materials, elements, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and their legal equivalents, and not limited to the foregoing description.

What is claimed is:

1. A wrist-type measurement system, comprising:
a measurement surface on which a user puts a wrist for measurement;
an opening configured on the measurement surface; and
a sensor configured under the measurement surface for measuring physiological information of the user on the wrist through the opening,
wherein the sensor is operable to scan the upper wrist surface along a scan path under the wrist to determine a measuring position in a non-contact mode and to move upwards through the opening to contact the wrist surface at the measuring position to measure the physiological information of the user in a contact mode.

2. The wrist-type measurement system of claim 1, further comprising a wristband which is worn on the wrist and coupled with the opening during the measurement.

3. The wrist-type measurement system of claim 2, wherein the wristband is coupled to the opening via magnetic effect.

4. The wrist-type measurement system of claim 3, wherein multiple magnetic components are configured along at least one side of the wristband for affixing the wristband to the opening.

5. The wrist-type measurement system of claim 2, wherein the wristband is coupled to the opening via locking mechanism.

6. The wrist-type measurement system of claim 5, further comprising a latch unit being configured on at least one side of the opening for locking the wristband when the wristband is coupled to the opening, and at least one control unit for controlling locking status of the latch unit.

7. The wrist-type measurement system of claim 2, wherein the wristband comprises an opening for defining a sensing area of the wrist when the wristband is worn on the wrist.

8. The wrist-type measurement system of claim 7, wherein one or more instruction signs are marked on the wristband for guiding the user to properly wear the wristband on at least one of the left and right wrists.

9. The wrist-type measurement system of claim 1, wherein the measurement surface has a front portion and a rear portion which is lower than the front portion.

10. The wrist-type measurement system of claim 9, wherein a slope is configured between the higher front portion and the lower rear portion.

11. The wrist-type measurement system of claim 9, wherein a recess is configured at the rear portion of the measurement surface.

12. The wrist-type measurement system of claim 1, further comprising a cantilever coupled to the sensor to drive the sensor to scan the wrist surface along the scanning path perpendicular to an artery under the wrist surface.

13. The wrist-type measurement system of claim 12, wherein the cantilever is operable to rotate around an axis and the sensor is configured at an end of the cantilever.

14. The wrist-type measurement system of claim 12, further comprising at least one leverage element being coupled between the cantilever and the sensor, wherein the leverage element is rotatable in relative to the cantilever to press the sensor towards the wrist.

15. The wrist-type measurement system of claim 12, further comprising a leverage unit coupled to the cantilever and rotatable in relative to the cantilever to lift the sensor, which is moveably configured at one end of the cantilever, towards the wrist.

16. The wrist-type measurement system of claim 1, further comprising an arm rest component for resting the user's arm.

17. The wrist-type measurement system of claim 1, further comprising a display holder operable for holding a display unit which is used to display the measurement result to the user.

18. The wrist-type measurement system of claim 1, wherein the sensor scans the wrist surface in a corresponding predetermined path based on which wrist is put on the measurement surface.

19. The wrist-type measurement system of claim 18, wherein the sensor is preset from an original position to an initial sensing position based on which wrist is put on the measurement surface, and further scans the wrist surface from the initial sensing position.

20. The wrist-type measurement system of claim 19, wherein the sensor is driven to move around the wrist within a predetermined sensing range from the initial sensing position.

21. A method for measuring physiological information of a user, comprising:
driving a sensor, which is positioned under a wrist of the user, to scan a skin area of the wrist along a scan path under the wrist;
determining a measuring position on the skin area based on a scanning result;
driving the sensor to move upwards to contact the skin surface at the measuring position; and
detecting the physiological information of the user at the measuring position by the sensor.

22. The method of claim 21, further comprising:
determining an optimal contacting force of the sensor on the wrist to detect the physiological information.

23. The method of claim 21, further comprising:
determining whether the wrist for measurement is a left or right wrist and driving the sensor to scan the wrist based on the determination result.

24. The method of claim 23, further comprising:
presetting the sensor from an original position to an initial sensing position based on which wrist is determined for measurement, and further scans the wrist surface from the initial sensing position along a corresponding predetermined path.

25. The method of claim 21, further comprising:
driving the sensor to scan the wrist in a direction perpendicular to an artery.

26. The method of claim 25, wherein the sensor is driven to scan around the wrist.

27. A method of applying a wrist-type measurement device to detect physiological information of a user, comprising:
wearing a wristband on a wrist of the user;
putting the wrist on the device and coupling the wristband to an opening of the device;
presetting a sensor of the device based on which wrist is put on the device; and
starting the measurement of the device.

28. The method of claim 27, wherein the step of presetting the sensor of the device based on which wrist is put on the device further comprises locating the sensor to a first initial sensing position when a left wrist is put on the device and locating the sensor to a second initial sensing position when a right wrist is put on the device.

29. The method of claim 27, wherein the step of coupling the wristband to an opening of the device further comprises coupling the wristband with the opening by magnet attraction.

30. The method of claim 27, wherein the step of coupling the wristband to an opening of the device further comprises locking the wristband with the opening mechanically.
